(19) United States
(12) Patent Application Publication
(10) Pub. No.: US 2008/0311147 A1
(43) Pub. Date: Dec. 18, 2008

(54) Rhabdoviral N-Fusion Proteins as Carrier for Foreign Antigens

(75) Inventors: Matthias J. Schnell, Harleysville, PA (US); Bernhard Dietzschold, Newtowne Square, PA (US)

Correspondence Address: PEPPER HAMILTON LLP, ONE MELLON CENTER, 50TH FLOOR, 500 GRANT STREET, PITTSBURGH, PA, 15219, US

(73) Assignee: Thomas Jeffeson University, Philadelphia, PA (US)

(21) Appl. No.: 11/568,006

(22) PCT Filed: Apr. 19, 2005
(86) PCT No.: PCT/US05/13298

§ 371 Date: Oct. 17, 2006

Related U.S. Application Data
(60) Provisional application No. 60/563,380, filed on Apr. 19, 2004.

(51) Int. Cl. A61K 39/00; C12N 15/00; A61P 31/00

(52) U.S. Cl. 424/199.1; 435/320.1; 424/236.1; 424/246.1; 424/201.1

(57) ABSTRACT

Rabies virus (RV) nucleoprotein (N) tightly encapsidates the genomic and antigenomic RNA thereby forming the ribonucleoprotein (RNP) complex. Antigens presented in a rigid and repetitive organization are sufficient to activate B cells to proliferate. In addition to the repetitive organization, it has been shown that RV N protein induces potent T-helper responses resulting in long-lasting and strong humoral immune responses against RV. The possibility to directly manipulate the genome of RV allows us to examine whether the immunogenicity of foreign antigens can be enhanced via incorporation into the RNP structure. A recombinant RV expressing an RV N-green fluorescent protein (GFP) fusion protein. The chimeric N-GFP fusion protein was efficiently expressed and incorporated into RV RNP and virions. Moreover, the recombinant RNP induces a strong humoral immune response against GFP in mice. In contrast, mice inoculated with GFP alone or a combination of wild-type RV RNPs and GFP did not trigger any GFP-specific humoral responses using the same immunization schedule. These results indicate the usefulness of RV-based vectors as killed vaccines against other infectious diseases.

### CROSS-REFERENCE TO RELATED APPLICATIONS

This application claims the benefit and priority of U.S. Provisional Application Ser. No. 60/563,380, filed Apr. 19, 2005 the contents of which are incorporated in this application in their entirety by reference.

### GOVERNMENT INTERESTS

The United States Government may have certain rights to this embodiment pursuant to work funded thereby at Thomas Jefferson University under Grant Nos. A149153, A144340, and A145097.

### BACKGROUND

Anthrax disease is caused by a binary A-B toxin system involving a binding component, Protective Antigen (PA), and Active components, Edema Factor (EF) and Lethal Factor (LF). Individually, none of the three toxin components causes disease. Toxification occurs following PA facilitated translocation of EF and/or LF. PA binds to a cell surface receptor and is cleaved by furin to its active form, PA63, which forms a heptameric structure capable of binding EF and LF. Once EF/LF are bound, the complex is internalized and acidification of the endosome triggers pore formation and translocation of the EF/LF to the cytosol where they act upon their substrates. Anthrax PA is so named because anti-PA antibodies confer a protective immune response to infection. PA-specific serum IgG has been shown to correlate to protection from challenge in animal models. PA is the immunodominant component of the currently licensed vaccine against anthrax. Sellman et al. have developed a novel toxin inhibitor, in which two mutations, K397D and D425K, have been introduced into PA63. This dominant negative mutant is able to participate in heptamerization, but is pore-formation and translocation deficient.

While antibiotics can be effective against B. anthracis, it is foreseeable that antimicrobial resistance could be engineered into future weapons-grade anthrax. Additionally, while antibiotics can eliminate the bacteria, they are ineffective against the toxins that have already been released after infection. Existing prophylactic and therapeutic strategies for combating anthrax are insufficient for several reasons. Historically, vaccination against anthrax was the first bacterial vaccination, which is successfully used as a live attenuated vaccine (Sterne strain) in animals. However, the residual toxicity disqualifies its use in humans. The human vaccine, Anthrax Vaccine Absorbed (AVA), contain the protective antigen (PA) component of the anthrax toxins as the major protective immunogen and is the only licensed vaccine in the U.S. However, AVA requires six doses in the first 18 months, with annual boosters. There is a need for a better dosing schedule for an Anthrax vaccine for humans, one with a well characterized and repeatable duration of protection, and a vaccine that minimizes potential severe side effects after vaccination.

As for most infectious diseases, the best strategy against Anthrax is an effective vaccine. However, the existing human vaccine against Anthrax experiences major problems due to the high incidence of toxicity and modest, transient humoral immune responses.

Within the Rhabdoviridae family of nonsegmented negative strand viruses having a bullet shape are the Ephemerovirus, vesiculovirus, and lyssavirus genera. The Rabies virus (RV) is a non-segmented negative-strand RNA virus within the Rhabdoviridae family and lyssavirus genera. The RV genome is about 12-kb in size and encodes five monocistronic RNAs encoding the nucleocapsid protein (N), phosphoprotein (P), matrix protein (M), the transmembrane glycoprotein (G), and the viral polymerase (L). The N protein, RV N encapsidates the viral RNA to form the ribonucleoprotein (RNP), which is the template for RNA transcription and replication by the viral polymerase-complex composed of the P and L proteins. The RV M bridges the RNP with the cytoplasmic domain (CD) of RV G in the host cell-derived viral membrane. The RV G mediates infection of the host cell.

Part of the immune protection from pathogens requires at least a strong B cell-mediated humoral response. Different virus-based expression systems suggest that viral particles or virus-like particles (VLPs) can serve as carriers of foreign B cell epitopes within the viral capsid or envelope protein of the viral particle. These “epitope” carriers resulted in strong B cell responses against the expressed foreign epitopes indicating their potential usefulness as carrier proteins. However, most of these approaches were restricted to incorporation of only short peptides that define epitopes due to the restricted cloning capacity within the carrier proteins.

Various features of carriers, or a combination of them, may be important to induce humoral responses against certain epitopes or antigens. Epitopes or antigens presented in a tightly, highly organized structure are sufficient to activate B cells to proliferate and induce IgM production. Linking of a strong T cell antigen to a B cell epitope enhances the B cell-specific immune response. An efficient presentation of antigens by antigen presenting cells (APC) plays an important role in the development of immunity. The stability of the antigen with the carrier in vivo aids in establishing a humoral response.

Recombinant live-viral vectors expressing foreign antigens efficiently induce potent cellular and humoral immune responses against the expressed antigens. RV can be used as an expression vector as shown with the model gene chloramphenicol acetyl transferase (CAT), which indicated that foreign genes can be expressed stably in RV. The possibility that RV could be used as a HIV-1 vaccine was tested, and it was shown in several studies that the expression of HIV-1 Env or Gag results in potent immune responses directed against HIV-1.

The immune responses induced by some vaccines, such as the current Anthrax vaccines, are not potent and not long-lasting and require frequent boosters to maintain protection. A carrier capable of incorporating epitopes, antigen, and whole proteins defining immunogenic subunits of an antigen could be used to promote a strong humoral response to the antigen in a subject or patient in need of immunization against such an antigen. These carriers could be used in vaccines and used to break immuno-tolerance, an important issue if immune responses against “self antigen” is advantageous such as for cancer vaccines or vaccines against spongiform encephalopathies (prion diseases like BSE).

### SUMMARY

Embodiments of carriers that can be used in vaccines can include compositions that express large subunits of antigens or whole antigen proteins linked or fused to a Rhabdovirus protein or variant of a Rhabdovirus viral protein. The expressed Rhabdovirus fusion proteins induce a humoral immune response in subjects receiving the fusion protein. These carriers could be used in vaccines and used to break immuno-tolerance. One embodiment is a composition that may include a rabies virus with a gene encoding an N or a G protein, or functional variants thereof, fused to a foreign antigen, such as, but not limited to, that from a toxin, a cancer; or a virus. In one embodiment of the composition, the rabies virus has an authentic N terminus.

One embodiment of a composition may include a rabies virus protein having at least a portion of an N protein or a G protein fused to a bacterial antigen, viral antigen, or cancer antigen, or variant thereof.

One embodiment of a method for immunizing an individual can include a gene from a rabies virus encoding a bacterial, viral, or cancer antigen, or variant thereof, fused to at least a portion of an N protein or a G protein wherein the rabies virus has an authentic N terminus.

One embodiment of a method for immunizing an individual can include administering to an individual an amount of at least one fusion protein expressed by a modified N gene or G gene of a rabies virus, the fusion protein comprises at least a portion of the N protein or G protein of the rabies virus linked to an immunogenic portion of an antigen protein, the amount of the fusion protein effective to elicit a B cell-mediated humoral immune response in the individual.

One embodiment of a composition includes a Rhabdoviridae virus nucleoprotein that is fused with a foreign antigen, epitope or their functional variants. The composition can be used as a vaccine to induce humoral immune responses against a foreign antigen, epitope, or their functional variant. The composition may include the rabies virus nucleoprotein fused to a viral or bacterial antigen such as but not limited to the Anthrax protective antigen, PA, or a functional variant of Anthrax PA. The compositions include a fragment of the antigen, such as the protective antigen of anthrax toxin or any other subunit or functional variant of the antigen which would have comparable immunogenic activity fused to the carrier Rhabdoviridae virus nucleoprotein and is capable of viral replication or assembly of the carrier virus. These fusion proteins are easy to purify and are able to induce strong humoral immune responses against the foreign protein, which does not occur when using the foreign antigen alone. Compositions of the present embodiment include but are not limited to chimeric RV Nfu-antigen proteins such as a chimeric N fusion protein of the anthrax protective antigen, RV Nfu-PA63 (SEQ ID NO: 28) or a functional variant of PA63 like Domain 4 of PA63, RV Nfu-D4 or its functional variants. The chimeric Nfu-antigen proteins may be expressed as additional proteins or functionally replace RV N.

One embodiment of a composition is a recombinant Rhabdovirus expressing a Rhabdovirus fusion protein of an antigen that is incorporated into Rhabdovirus RNP and its virions. Preferably the genome of the recombinant Rhabdovirus expressing the fusion protein of the antigen includes a nucleoprotein gene N, that expresses a nucleoprotein with an N-terminus which allows the incorporation of the expressed fusion protein of the antigen into the Rhabdovirus RNP. More preferably the recombinant Rhabdovirus is RV, whose genome includes a nucleoprotein gene N that expresses a nucleoprotein with an N-terminus that allows or directs the incorporation of the expressed fusion protein of the antigen into RV RNPs. Most preferably the recombinant RV genome includes a nucleoprotein gene N that expresses a nucleoprotein with an N-terminus which allows the incorporation of the expressed fusion protein of the antigen, Nfu-antigen, into the RPN, and wherein the Nfu-antigen protein is expressed from an extra gene, between the G and L genes of the RV genome.

In various embodiments, the nucleoprotein N of the recombinant Rhabdovirus is a B cell antigen and tightly encapsulates the Rhabdovirus into an RNAase resistant RNP core.

Various embodiments may be used in pharmaceutical compositions and vaccines to induce an improved immune response in a subject to the antigen using a viral capside (RNP) or virion as a carrier for the foreign antigens or their functional equivalents. An example of a carrier includes the rabies virus and the antigen may include but is not limited to the Anthrax protective antigen PA or a functional variant of Anthrax PA. Recombinant Rhabdovirus expressing a Rhabdovirus antigen fusion protein, such as recombinant RV RNP containing Anthrax PA, can be used to immunize test subjects like mice and humoral immune responses can be followed by ELISA assays, proliferation assays, and in vitro and in vivo toxicity neutralization assays. Existing vaccines or recombinant antigens like PA63 may serve as controls. Toxin neutralization can be measured in macrophage cytotoxicity assays or through mouse lethal toxin challenges. Exposure of the test subjects to a virus or bacterial challenge, for example an Anthrax challenge experiment, can be used to evaluate the protection provided by the carrier and antigen portions of the expressed fusion proteins as vaccines for use against the virus or bacteria in other subjects or for humans.

In one embodiment killed Rhabdovirus particles, like rabies virus particles, are used as a vaccine carrier that display antigen proteins or their functional variants on the surface of the Rhabdovirus particles. Antigen proteins, or their functional variants, may include but are not limited to viral and bacterial antigens such as anthrax PA63 or D4 proteins. Different viral and bacterial antigen proteins, such as but not limited to anthrax PA63 or D4 proteins, may be incorporated into the glycoprotein of the rhabdovirus virions. Recombinant RVs expressing these antigen proteins can be used to prepare RV particles with the antigen protein incorporated into the glycoproteins on the surface of the virions. Recombinant virions can be purified and used to immunize and challenge test subjects such as but not limited to mice and vaccination/challenge experiments used to screen for recombinant virions useful for vaccines for further testing in other mammalian subjects or in humans.

One embodiment is a composition that includes a Rhabdovirus nucleoprotein having an authentic N terminus fused to an antigen protein. The nucleoprotein having the authentic N terminus fused to the antigen protein may be expressed by a recombinant Rhabdovirus. Preferably the nucleoprotein having the authentic N terminus fused to a antigen protein is expressed by the SPBN-Nfu-antigen virus. Even more preferably the Nfu-antigen protein expressed by a recombinant SPBN-Nfu-antigen virus in cells. Another embodiment is a method of screening recombinant RV RNPs that includes immunizing test subjects with recombinant RV RNP's expressing Nfu-antigen, measuring humoral immune responses, macrophage cytotoxicity assays, or mouse lethal toxin challenge of the test subjects to the immunization; and selecting a chimeric fusion protein, like Nfu-antigen, which may be used in the development of vaccines against the antigen.

Another embodiment is a method of making a non-segmented negative-stranded RNA virus entirely from cDNA which includes intracellular expression of antigenomic RNA in cells also expressing the viral proteins for formation of an active RNP complex. Preferably the cellular expressed viral proteins include the nucleoprotein (N), the phosphoprotein (P), and the viral polymerase (L).

Advantageously, immune responses, which are not induced by the use of the antigen itself can be induced in the present embodiment where the Rhabdovirus nucleoprotein is fused to the foreign antigen. Whole proteins can be presented to the immune system in such a way as to induce strong B cell responses. The induced immune response to the protein is expected to be long lasting and improve the protection provided by vaccines. An anthrax vaccine that was longer lasting would reduce the number of immunizations for subjects, improve patient compliance, and provide greater patient protection between inoculations.

The present compositions and or methods may be used to break immuno-tolerance, when the animal is unable to immunologically respond to a particular antigen, where the development of an immune responses against “self antigen” can be beneficial for cancer vaccines.

RV N may be used as a carrier protein for foreign epitopes as well as antigen proteins and their functional variants. Advantageously, RV N primes T cells and induces RV N-specific antibodies. From the strong humoral immune responses against RV N it is expected that RV Nfu-antigen protein is well presented by antigen presenting cells (APCs). Advantageously, RV RNP is highly resistant to proteolytic enzymes and therefore should also promote the long-term presentation of a foreign antigen to the immune system. Large amounts of these proteins can be produced. No adjuvant is needed. The compositions may be used in vaccines, antibody production, as well as in the purification and production of recombinant proteins which are difficult to produce or purify. One embodiment of a composition is a human Anthrax vaccine that includes a viral capsid (RNP) or virion as a carrier for the Anthrax protective antigen PA or a portion of the Anthrax protective antigen. Foreign antigens presented by recombinant rabies virus (RV) ribonucleoprotein (RNP) or virions induce humoral responses and can induce potent humoral responses. RV-based vectors are also excellent to induce strong and protective B-cell responses against Anthrax. RV mediated T-helper response(s) and the presentation of Anthrax protective antigen (PA) to the immune system in a highly organized manner is expected to be superior to the use of recombinant Anthrax PA alone.

### DESCRIPTION OF THE DRAWINGS

The file of this patent contains at least one drawing/photograph executed in color. Copies of this patent with color drawing(s)/photograph(s) will be provided by the Office upon request and payment of the necessary fee.

In part, other aspects, features, benefits and advantages of the various embodiments of the present embodiment will be apparent with regard to the following description, appended claims and accompanying drawings where:

FIG. 1(A-I) illustrate recombinant RVs expressing N antigen fusion proteins or recombinant RV's expressing G-antigen fusion proteins, wherein the ectodomain of the G in the recombinant RV's expressing the G-antigen fusion protein is modified. FIG. 1(A) (BNSP-Nfu) an RV vector (SEQ ID NO: 24), which allows the expression of an RV N fusion protein, was constructed by deleting the RV N stop codon and introducing the two single restriction sites BsiWI and NheI to give BNSP-Nfu. FIG. 1(B) (BNSP-Nfu-GFP (SEQ ID NO:29)), the vector site of BNSP-Nfu was the target to introduce the coding region of GFP (BNSP-Nfu-GFP). FIG. 1(C) (SPBN-Nfu-GFP), is an RV-based vaccine vector expressing an RV Nfu-GFP (SEQ ID NO:29) fusion protein with an extra gene that was constructed by introducing the gene encoding RV Nfu-GFP (SEQ ID NO: 29) between a new RV transcription unit in SPBN resulting in the virus vector SPBN-Nfu-GFP. FIG. 1(D) SPBN-GFP, the SPBN-GFP vector schematic shows a RV vector expressing GFP. FIG. 1(E) (SPBN-Nfu-HC50), construction of recombinant RV expressing RV-Nfu HC50 (botulinum neurotoxin domain HC50) fusion protein. FIG. 1(F) BNSP-Nfu-HC50, a recombinant RV where RV N protein is replaced by the RV-Nfu-HC50 protein. FIG. 1(G) SPBN-Nfu-antigen.

FIG. 2A illustrates expression of Anthrax PA63 by RV. BSR cells were infected with an empty RV vector (SPBN) or RV expressing PA63 as an additional gene (SPBN-PA63). Cells were fixed and immunostained with an antibody directed against RV N protein (α-RV-N) or anthrax PA63 (α-PA63). FIG. 2B illustrates recombinant Nfu-GFP (SEQ ID NO:30) fusion protein co-localizes with RV N(SEQ ID NO:26). BSR cells were infected with virus vector SPBN-GFP (panels A, A′) or SPBN-Nfu-GF (panels B and B′). Cells were fixed two days later and immunostained with a monoclonal antibody directed against GFP (panels A′ and B′) or an antibody specific for RV N (SEQ ID NO:26) protein (panel A and B). The results indicated that wild-type GFP has a diffuse distribution within the infected cell whereas the Nfu-GFP (SEQ ID NO:30) fusion protein localizes similar to RV N (SEQ ID NO:26) protein. FIG. 2C illustrates expression of chimeric RV Nfu HC50 fusion protein by RV. BSR cells were infected with an empty RV vector (SPBN) or RV SPBN Nfu Hc50 expressing Nfu-HC50 fusion protein as an additional gene (SPBN-N-HC50). Cells were fixed and immunostained with an antibody directed against RV N protein (SEQ ID NO:26) (α-RV-N) or BoNT/A (α-BoNT/A).

FIG. 3. Characterization of recombinant RV expressing Nfu-GFP (SEQ ID NO:29). Panel A: To analyze if the recombinant Nfu-GFP protein (SEQ ID NO:30) is associated with RV RNPs, RNPs from SPBN-Nfu-GFP infected cells were purified by CsCl density centrifugation. A sharp band, which fluoresces green during exposure to light, can be seen. Panel B: Twelve fractions were collected from the CsCl gradient, dialyzed, and resolved by SDS-PAGE. Probing a Western blot with a RV N-specific antibody detected the highest concentration of RNPs in fraction 9, similar to the results seen for wild-type RNPs (not shown). In addition, a GFP-specific antibody detected equal amounts of Nfu-GFP (SEQ ID NO:30) and N (SEQ ID NO:26) in all fractions, further indicating incorporation of Nfu-GFP (SEQ ID NO:30) into RV RNPs. Panel C: Virions from BSR cells infected with SPBN or SPBN-Nfu-GFP were purified over 20% sucrose, separated by SDS-PAGE, and analyzed by Western blotting. The results indicate that the Nfu-GFP (SEQ ID NO:30) protein is incorporated into RV virions. Panel D: Purified RV RNPs from cells infected with a recombinant RV expressing N-HC50. RNPs from cells infected with SPBN-N-HC50 were purified by CsCl and separated by SDS-PAGE. Western blot analysis with a RV N specific antibody detects two proteins migrating at the expected size for RV N (SEQ ID NO:26) and N-HC50. N-HC50 is also detected by a BoNT-specific antibody.

FIG. 4. One-step growth curve of RV vectors. BSR cells were infected with SPBN or SPBN-Nfu-GFP at a MOI of 0.01. Aliquots of culture supernatants were collected at indicated timepoints post-infection and viral titers were determined in duplicate on BSR cells.

FIG. 5. Comparison of GFP immunogenicity using different approaches. Groups of five mice were primed with RNPs containing the RV N-GFP fusion protein (SEQ ID NO:30) (Nfu-GFP, circles), a mixture of recombinant GFP protein and wild-type RV RNPs (N+GFP, squares), or GFP protein only (GFP only, triangles). Because no seroconversion was detected after priming, the data points for the prime (first immunization) are not shown in this graph. The same antigens were applied in a second boost, (red solid lines) and third boost (green solid lines) immunization. The upper panel show the RNP-specific ELISA and the lower panel the GFP-specific ELISA.

FIG. 6 illustrates construction of recombinant RVs expressing RV-N Anthrax-PA fusion proteins. FIG. 6 shows the three different RVs expressing Anthrax PA63 (SPBN-Nfu-PA63) (Nfu-PA63 SEQ ID NO:27) or the domain 4 of Anthrax PA63 (D4) (SPBN-Nfu-D4) as an additional gene. BNSP-Nfu-D4 is a recombinant RV where the RV N protein is replaced by the RV-Nfu-D4 protein.

FIG. 7. Rabies virus recovery system. BSR cells (a BHK cell clone) stably expressing bacteriophage 17 RNA polymerase are transfected with four different plasmids (circles), three plasmids encoding the RV virus viral proteins [nucleoprotein (N), phosphoprotein (P), polymerase (L)], and one encoding the anti-genomic RNA of the respective recombinant RV. All plasmids contain the promoter for the T7 RNA polymerase. A successful recovery uses the encapsidation of the anti-genomic RNA into the nucleoprotein building the ribonucleoprotein (RNP, vg+ see figure) and the subsequent replication by the RV polymerase complex (P and L protein) to the genomic RNP (vg−). The genomic RNP serves as a template for expression of all viral protein N, P, M (matrix), G (glycoprotein), L, and the assembly, budding and release of infectious RV from the cell membrane.

FIG. 8. Is an illustration of an immunization schedule.

FIG. 9. Chimeric RV G Anthrax PA protein. (A) pSSTMT contains the RV G signal sequence (SS), two single restriction sites (SpeI/HpaI) to introduce the PA sequences, the RV G transmembrane (TM), and cytoplasmic domain (CD). The flanking BsiWI/NheI sites allow the subcloning of these sequences to the RV expression vector pSPBN if surface expression is confirmed in transient expression system. (B): Possible constructs: RVG-PA63, full-length Anthrax PA63 in pSS-TMT resulting in PA63 with an aminoterminal RV G SS and a carboxyterminal TM and CD of RV G. The four other constructs are based on chimeric RV G/PA domain four (D4) fusion proteins containing portions of the RV G ED, RVG-D4-439, D4+full length ED; RVG-D4-210 210 aa of ED; RVG-D4-127 127 aa of ED; RVG-D4-51 (SEQ ID NO:34) 51 aa of RV GED. The potential RV G N-glycosylation sites are indicated (branched symbols).

FIG. 10(A) and FIG. 10(B) mice were immunized with recombinant RNP's containing BoNT A HC50. A first boosted was given 10 days later using the same antigens used for priming. A second boost was given 10 days after the first boost. Serum dilutions used in ELISA were 1:40; 1:120; 1:360; 1:1080 (left to right). ELISA for seroconversion of immunized mice against the botulinum neurotoxin, HC50. Two groups of 5 6 week old female Swiss Webster mice were immunized i.m. with 5 μg of recombinant RNPs derived from SPBN-N-HC50 (RNP-BotA), or 5 μg RNPs containing not HC50 as a control (RNP-X). Neither a humoral immune response against RV N or seroconversion against HC50 was detected in any of the two groups of mice after priming. However, the first boost with the same antigens as used for priming resulted in a strong RV N specific immune response in both groups of mice immunized with RNP (FIG. 10(A) RV RNP ELISA Reactivity, red bars (post 1^{st} boost)). In addition, an HC50-specific humoral response was detected in the sera of mice immunized with N-HC50 (FIG. 10(B) RV Botulinum ELISA Reactivity, red bars (post 1^{st} boost)). As illustrated in FIG. 10(B), the second boost (blue bars) further enhanced the RNP specific responses but also resulted in a dramatic increase in HC50 specific responses in the case of RNP-N-HC50 immunized mice. Again, no HC50-specific immune response was detected in mice immunized with the control RNP (FIG. 10(B) RV Botulinum A Hc50 ELISA Reactivity, RNP-X, blue bars (2 boost)).

FIG. 11(A) Chimeric RV G Anthrax PA protein, cartoon of the RV G glycoprotein signal sequence (SS), the RV G ectodomain (ED), transmembrane domain (TM), and cytoplasmic domain (CD) is indicated. FIG. 11 (B-F) different construct containing the RV G sequences fused to Anthrax PA 63 D4 were constructed. All constructs contain the RV SS, TM and CD and different portions of the RV G ED (0 to 439 amino acids, B-F).

FIG. 12. Surface expression of recombinant PA on the cell surface. Forty-eight hours after transfection, cells were fixed with 4% paraformaldehyde, immunostained using a polyclonal rabbit antibody directed against Anthrax PA and FITC conjugated donkey anti-rabbit and analyzed by fluorescence microscopy. Permeabilized control cells were used to ensure the expression of the recombinant proteins.

FIG. 13. ELISA for seroconversion of immunized mice against RV G. Three groups of 5 Swiss Webster mice were immunized with recombinant virions containing Anthrax PA63 i.m (D4-E51 killed), wild-type virions (killed SPBN), or the live RV vector expressing D4-E51 (3×10⁶ ffu, D4-E51 live) at week zero and boosted the mice three weeks after prime with the same vaccine. ELISA results are listed for different sera dilution pooled from five mice of each group two weeks after prime (post-prime) and two weeks after boost (post-boost).

FIG. 14. ELISA for seroconversion of immunized mice against Anthrax PA. Three groups of 5 Swiss Webster mice were immunized with recombinant virions containing Anthrax PA63 i.m (D4-E51 killed), wild-type virions (killed SPBN), or the live RV vector expressing D4-E51 (3×10⁶ ffu, D4-E51 live) at week zero and boosted the mice three weeks after prime with the same vaccine. ELISA results are listed for different sera dilution pooled from five mice of each group two weeks after prime (post-prime) and two weeks after boost (post-boost). The data indicate that one single inoculation with live or killed RV virions containing the chimeric RV G/PA 63-D4 protein induce Anthrax PA-specific seroconversion. A second inoculation increased these responses to high titers.

### DETAILED DESCRIPTION

Before the present compositions and methods are described, it is to be understood that they are not limited to the particular molecules, compositions, methodologies or protocols described, as these may vary. It is also to be understood that the terminology used in the description is for the purpose of describing the particular versions or embodiments only, and is not intended to limit the scope of the disclosure which will be limited only by the appended claims.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of various embodiments, the preferred methods, devices, and materials are now described. All publications mentioned herein are incorporated by reference. Nothing herein is to be construed as an admission that the various embodiments are not entitled to antedate such disclosures.

“Optional” or “optionally” means that the subsequently described event or circumstance may or may not occur, and that the description includes instances where the event occurs and instances where it does not.

Foreign antigens, viral or bacterial, presented by recombinant Rhabdovirus ribonucleoprotein (RNP) or virions may be used to induce potent humoral responses when administered to subjects. These Rhabdovirus-based vectors may induce strong and protective B-cell responses against the antigen. Chimeric Rhabdovirus Nfu-antigen fusion proteins and pharmaceutical compositions including them may be used as strong B-cell antigens. The genome of a Rhabdovirus may be modified to construct a recombinant Rhabdovirus expressing an Nfu-antigen fusion protein or an Nfu-antigen variant fusion protein, the antigen variant having the function of the antigen. The chimeric Nfu-antigen or Nfu-antigen variant proteins can be expressed as additional proteins to functionally replace the Rhabdovirus N protein or as a mixture with Rhabdovirus N protein.

Recombinant Rhabdovirus RNPs containing the antigen or its functional variant (immunogenic variant) can be used to immunize subjects like mice or other mammals including humans, and the humoral responses of the subjects can be followed by ELISA assays. Existing vaccines, antibodies, or recombinant antigens may be used as controls. In the case of bacterial toxins, toxin neutralization can be measured in macrophage cytotoxicity assays, and in subject lethal toxin challenge. These tests may be used to screen which chimeric Rhabdovirus antigen fusion proteins are useful for general use in subjects or for further development.

Killed or live Rhabdovirus virions or particles may be used as an efficient vaccine carrier to display antigens or their functional variants. Various viral, bacterial, and cellular antigens can be incorporated into the glycoprotein of the Rhabdovirus virions. The genome of the Rhabdovirus, and preferably the G gene, can be modified to construct recombinant Rhabdovirus virions expressing the antigen in the glycoprotein of the recombinant Rhabdovirus virion. Recombinant virions may be purified and used to immunize and challenge subjects like mice as well as other mammals including humans, and the humoral responses of the subjects can be followed by ELISA assays. Existing vaccines, antibodies, or recombinant antigens may be used as controls. In the case of bacterial toxins, toxin neutralization can be measured in macrophage cytotoxicity assays, and in subject lethal toxin challenge. These tests may be used to screen which chimeric Rhabdovirus antigen fusion proteins are useful for general use in subjects or for further development.

GFP green fluorescent protein, is an example of a protein that RV N protein can serve as a carrier of a “whole” foreign protein. The Nfu-GFP fusion protein was efficiently incorporated in RV RNPs and virions. As demonstrated, humoral responses were only detected when GFP was presented by RV RNPs and no B cell responses were detected even after three immunizations with recombinant GFP.

From the findings in the examples with GFP it is reasonable to expect that foreign antigens or their functional variants presented by the ribonucleoprotein (RNP) of recombinant Rhabdoviruses, preferably Lyssaviruses, and most preferably the rabies virus (RV) can induce potent humoral responses, and that Rhabdovirus-based vectors are also excellent to induce strong and protective B-cell responses against foreign proteins. It is expected that Rhabdovirus mediated T-helper response(s), and preferably RV mediated T-helper response(s) and the presentation of foreign antigens or their immunogenic/functional variants to the immune system in a highly organized manner is superior to the use of recombinant protein alone.

Rabies virus ribonucleoprotein is a carrier for foreign proteins. RV RNPs are structurally stable, easy to purify and excellent B cell antigens and therefore a good source for protein production and as a vaccine. A segment of the carboxy terminus of RV N (contained in RNP) is cleaved by trypsin treatment suggesting that this portion of RV N is probably not bound to the viral RNA and therefore might be a good site at which to add foreign peptides. The use of the RV N carboxy terminus is supported by the finding that the deletion of 18 amino acids from the C terminus of New Castle Disease virus (NDV) nucleoprotein, another negative-strand RNA virus, did not affect the production of infectious NDV.

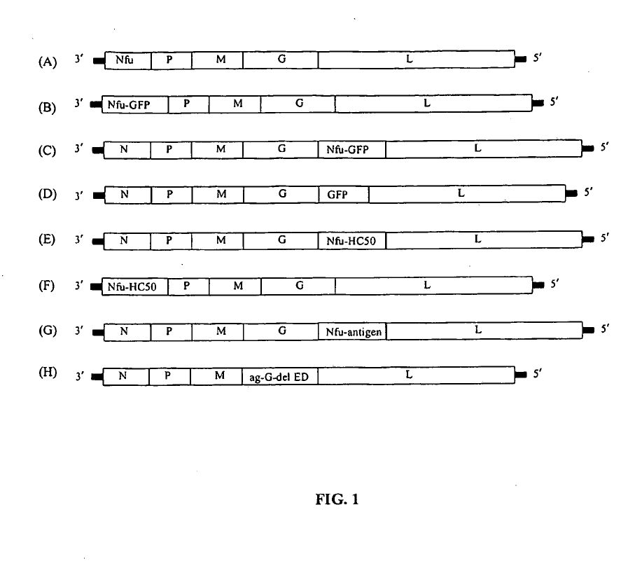

An RV vector can be generated and utilized (BNSP-Nfu virus, FIG. 1A) to construct a recombinant Rhabdovirus like RV that allows the addition of foreign sequences fused to the RV N protein. The plasmid pBNSP-Nfu can be constructed in such a way that the stop codon of N is deleted and two restriction sites introduced, which makes it possible to add a foreign amino acid sequence at the carboxyterminus of RV N. In the process of deleting the RV N stop codon, four foreign amino acids were added to the C-terminus of the RV N due to the BsiWI and NheI restriction sites. It was possible to recover the BNSP-Nfu virus indicating that there are no stringent sequence requirements for the RV N protein C-terminus for RNA transcription, replication, or viral assembly. In the next step the coding sequence of GFP was cloned, as a model protein, into BNSP-Nfu resulting in pBNSP-Nfu-GFP (FIG. 1B). Multiple attempts to recover BNSP-Nfu-GFP virus were unsuccessful; without wishing to be bound by theory this suggests that the Nfu-GFP fusion protein may have failed to functionally replace wildtype RV N in the viral life cycle. Expression of the Nfu-GFP protein from an extra gene, between G and L, from the RV genome (FIG. 1C SPBN-Nfu-GFP virus) did allow recovery of recombinant SPBN-Nfu-GFP virus. Without wishing to be bound by theory, this may demonstrate that an authentic N terminus still allows the incorporation of the recombinant protein into RV RNP's.

In Rhabdoviruses the authentic N-terminus, i.e., the amino terminus, is the N terminus of the Rhabdovirus N protein, which can be the N terminus of the N fusion protein, Nfu-antigen. This part probably contains the sequence that binds to the viral RNA, which is part of the RNP. The N protein forms a complex with the RNA (RNP) allowing encapsidation of the RNP and the Nfu of the fusion protein, Nfu-antigen protein, does the same. The other viral proteins do not form a complex with the RNA but are bound to the RNA, in a smaller molecular ratio. For RV, the fusion protein Nfu-antigen (N protein+foreign protein) can be expressed from different sites within the genome, and foreign genes for the expression may be placed between RV genome genes such as between N and P, P and M, G and L.

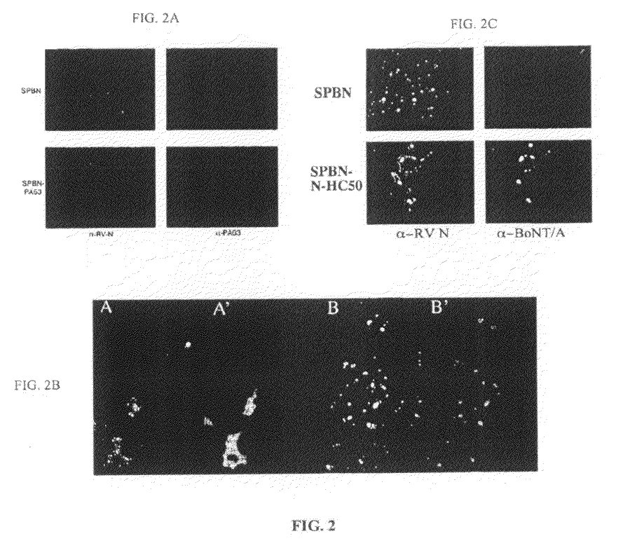

RV N-GFP fusion protein co-localizes with RV N within infected cells. The recombinant Nfu-GFP protein (SEQ ID NO:30) expressed by SPBN-Nfu-GFP virus in BSR cells were analyzed (SPBN-Nfu-GFP virus). An RV virus expressing wildtype GFP as an extra gene served as a control virus (SPBN-GFP virus, FIG. 1D As shown in FIGS. 2 A and B, immunostaining with an RV anti-N-specific antibody shows RV N mainly contained in intracellular inclusion bodies, which have been proposed as sites of viral replication and transcription (FIGS. 2 A and B). Likewise, a large amount of the recombinant Nfu-GFP fusion protein expressed by SPBN-Nfu-GPP virus, which showed a similar localization as RV N (FIG. 2 B′), indicating that the RV N portion of the fusion protein is able to redirect the protein to the sites of RV replication. In contrast, in SPBN-GFP virus infected BSR cells immunostaining specifically for GFP resulted in the detection of a diffuse distribution of GFP within the infected cell (FIG. 2 A′). Similar localization for RV N and recombinant Nfu-antigen fusion protein expressed by SPBN-Nfu-Antigen virus would be expected.

The content of Nfu-GFP (SEQ ID NO: 30) in virions was about a third of that detected for RV N (SEQ ID NO: 26). To isolate large quantities of the recombinant RNPs for immunogenicity studies and to analyze if Nfu-GFP (SEQ ID NO: 30) is incorporated into RV RNPs, BSR cells were infected with SPBN or SPBN-Nfu-GFP. The cells were lysed after 48 hours and RNPs purified by a CsCl density gradient. The recombinant RNPs containing Nfu-GFP (SEQ ID NO: 30) fluoresce green when exposed to long wavelength UV light. In addition, the analysis of the recombinant RNPs showed a similar distribution of the RNPs in the collected fraction as observed for RV wild-type RNPs.

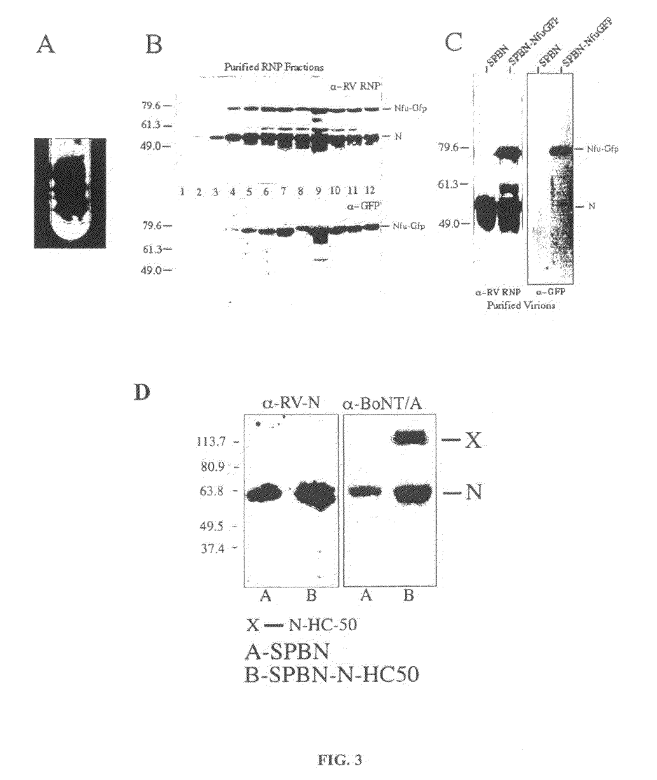

The RV N serves as carrier to incorporate GFP into RV RNPs. The experiments by the inventors suggest that RV N moiety of the Nfu-GFP (SEQ ID NO: 30) protein is able to direct a foreign protein to the sites of RV replication. To analyze if the recombinant RV Nfu-GFP (SEQ ID NO: 30) protein is incorporated into RV RNP, lysates of SPBN-Nfu-GFP or SPBN infected cells were subjected to isopycnic centrifugation, which revealed that RNPs from SPBN or SPBN-Nfu-GFP formed a sharp band at identical densities. The RNP band derived from SPBN-Nfu-GFP virus-infected cells fluoresced green during exposure to light indicating the presence of functional GFP in the RNP complex (FIG. 3, panel A). When fractions were collected from each CsCl gradient, and analyzed by Western blotting using a RV anti-N-specific antibody, the analysis revealed that the GFP immunoreactivity completely overlapped with the RV immunoreactivity, clearly indicating incorporation of Nfu-GFP (SEQ ID NO: 30) into the RV RNP complex (FIG. 3, panel B). Based on structural similarity between RV and other Rhabdoviruses, it is reasonable to expect that various Rhabdovirus RNP's will incorporate Nfu-antigen proteins into the Rhabdovirus complex; the methods described supra may be used to determine the incorporation.

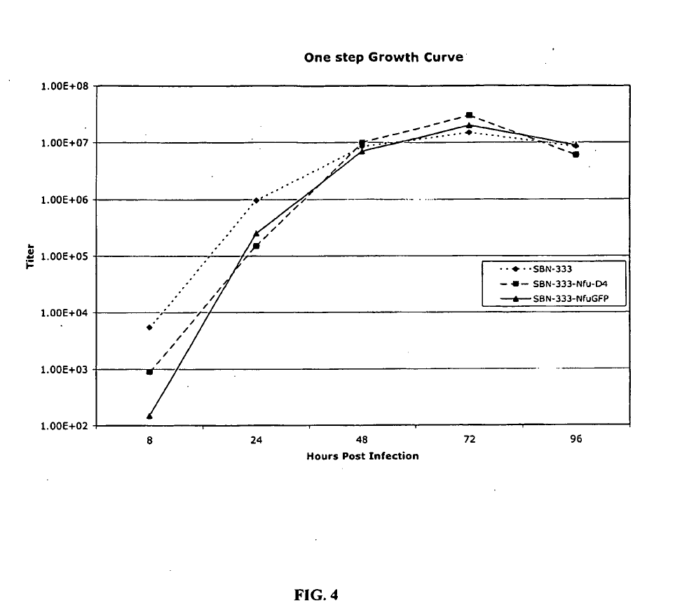

Recombinant Nfu-GFP RNPs may be incorporated into RV virions and do not affect viral growth in vitro. To determine if the large amount of Nfu-GFP (SEQ ID NO: 30) expressed from the “extra” gene in the recombinant RNPs might prevent or interfere with their packaging of RV virions. To analyze if recombinant RNPs containing Nfu-GFP (SEQ ID NO: 30) were also incorporated into RV virions, cells were infected with SPBN virus or SPBN-Nfu-GFP virus at an MOI of 1 for 72 hours and supernatants were purified over 20% sucrose. Virion proteins were separated by SDS-PAGE, transferred to a PVDF-Plus membrane, and analyzed by Western blots using antibodies directed against RV N or GFP. The results in FIG. 3, panel C show one band of 50 kD as expected for RV N (SEQ ID NO: 26) for both viruses. However, an additional band of about 80 kD was detected in virions from SPBN-Nfu-GFP infected cells. Immunoblotting with a monoclonal antibody directed against GFP also detected a protein migrating at 80 kD confirming that the detected 80 kD protein was Nfu-GFP (SEQ ID NO:30). Using a Molecular Imager to measure the amount of Nfu-GFP (SEQ ID NO:30) in RNP, it was determined that Nfu-GFP (SEQ ID NO:30) was about 30% of wild-type N in recombinant RV virions, which was similar in amount to the expression levels seen in SPBN-Nfu-GFP infected cells. As listed above, the major concern with this approach was that the recombinant fusion protein might interfere with the viral life cycle just resulting in low or unstable viruses. This result confirmed that recombinant RVs are not excluded from viral assembly but that there was still the possibility that viral titer would be affected. Because immunostaining did not indicate any reduction in production of viral protein (FIGS. 2 A and B), analysis of the viral replication cycle was performed by a one step growth curve. BSR cells were infected with SPBN or SPBN-Nfu-GFP at an MOI of 5 and samples were collected at different times as indicated in FIG. 4. The results demonstrate that both viruses show approximately the same viral titer at all 5 time-points. Therefore, neither the larger genome of SPBN-Nfu-GFP nor the expression of two nucleoproteins had any major impact on viral replication or assembly. These results further confirm the high flexibility of RV genome to carry new proteins with minimal effects on their life cycle. Recombinant Rhabdoviruses expressing Nfu-antigen (fusion) proteins are preferably not excluded from viral assembly, do not interfere with the viral life cycle, or viral assembly.

Recombinant RV RNPs containing GFP are highly immunogenic in mice. As indicated above, RV RNPs are highly immunogenic. A variety of other Rhabdovirus RNP's are also highly immunogenic, and it is reasonable to expect that Rhabdovirus RNP's containing Nfu-antigen fusion proteins are also immunogenic. In a non-limiting example, to study the immunogenicity of recombinant RNPs containing GFP, groups of five six-week-old female C57 mice were immunized i.m. with 5 μg recombinant RNPs derived from SPBN-Nfu-GFP virus, or 2 μg GFP alone. To examine the immunostimulating effect of RNP itself, 5 control mice were inoculated with a mixture of 2 μg recombinant GFP and 5 μg RV wild-type RNPs. Mice were boosted at weeks two and five. Neither a humoral response against RV N or seroconversion against GFP was detected in any of the three groups of mice after priming. However, the first boost with the same antigens as used for priming resulted in a strong RV N-specific immune response in both groups of mice immunized with RNP (FIG. 5, RNP ELISA, red lines s(quares and circles)). In addition, a GFP-specific humoral response was detected in the sera of mice immunized with Nfu-GFP (FIG. 5, GFP ELISA, red circles). The second boost resulted in a dramatic increase in GFP-specific responses in the case of RNP-Nfu-GFP-immunized mice (FIG. 5, GFP ELISA green circles, green solid lines) and also enhanced the RNP-specific responses (RNP ELISA, green squares and circles, green solid lines). No GFP-specific immune response was detected in mice immunized with the mixture of GFP/RNPs or GFP alone (FIG. 5, GFP ELISA squares and triangles). These data indicate that immunization with an RNP-Nfu-protein greatly enhanced the immune responses directed to the foreign protein (GFP) or other antigen.

To assess if the observed humoral response depends on T cell help, a group of 5 mice was CD4+ T cell-depleted, whereas another group of non-depleted mice served as a control. All mice were immunized i.m. with Hong Nfu-GFP (SEQ ID NO:30) and given boosts at day 12 with the same antigen. Mice were bled at days 0, 4, 8, 12, 16, 20, and 24 and analyzed by ELISA for RNP, or GFP-specific IgM. No GFP-specific IgM was detected for any group at any time point. However, low, but significant anti-RNP IgM antibodies were detected in the non T-cell-depleted mice four days after the immunization boost (data not shown). These results indicate that the detected anti-RNP and anti-GFP antibody production depended on T-cell help. The enhanced immune responses directed to the foreign protein (GFP) in Nfu-GFP (SEQ ID NO:30), should also be present in fusion proteins generally such as Rhabdovirus Nfu-antigen, and more preferably in RV Nfu-Anthrax PA fusion proteins (infra).

Long-lasting humoral immune responses depends on both the activation of B cells and T cell helper responses. The RV N is an excellent B cell antigen and ˜1,800 RV N molecules tightly encapsidates the RV genome into an RNAase resistant RNP core. Antigens presented in such a repetitive, tightly, and highly organized structure are often sufficient to activate B cells to proliferate and induce an IgM response. This has been shown for antigens like RV and VSV G, which are highly immunogenic if presented by the viral particle and which do not activate B cells as a soluble antigen. This seems to be different for a highly organized antigen such as RV RNP. The inventors have determined that GFP presented to B-cells by the recombinant RNPs acts as T cell-dependent antigen because no significant anti-GFP IgM production was detected in CD4⁺ T-helper cell-depleted mice. In addition, anti-N IgM production against RV N was not detected in these mice, suggesting that RV RNPs, in general, act as a T cell-dependent antigen. Mechanisms other than T-helper cell-independent responses may be involved in the observed strong B cell response of antigens presented in such a highly ordered structure as RNPs.

A ribonucleocapsid complex refers to the ribonucleocapsid and ribonucleoprotein deriving from a genome of a virus, preferably an RNA virus, more preferably a single stranded or negative stranded RNA virus, which has been purified from the virus or produced recombinantly. This may include the N-protein component of the ribonucleoprotein as well as other proteins which can also be used in combination with a selected antigen to produce an enhanced immune response. The ribonucleocapsid complex can originate or be derived from a selected virus, preferably a negative strand RNA virus, and can be further produced and modified by recombinant or other bioengineering techniques. Negative strand viruses are preferred in the practice of the various embodiments and may include but are not limited to Rhabdoviruses which are a family of viruses with a non-segmented negative strand RNA genome and whose virions are in the shape of a bullet.

The Rhabdoviruses include the Lyssavirus, Ephemerovirus, and Vesiculovirus viruses, and the Lyssaviruses include the Rabies virus. The Rhabdoviruses, and those with modified genes in their genome; that may be used in the present embodiment are negative strand virus having a RNA-ase resistant ribonucleoprotein (RNP) core and a glycoprotein covering the virion surface. The nucleoprotein that makes up the RNP and encases the viral RNA preferably induces T helper responses and that fusion proteins of the antigen formed with the nucleoproteins are strong B cell antigens. The glycoprotein gene for expressing the glycoprotein covering the surface of the virion is capable of being modified by introduction of sequences for expression of chimeric glycoprotein-antigen proteins on the surface of the modified Rhabdovirus virion. In some embodiments the glycoprotein gene for expressing the glycoprotein covering the surface of the virion is modified by removing portions of the ectodomain sequence, resulting in the expression of chimeric glycoprotein-antigen proteins with a modified ectodomain on the surface of the modified Rhabdovirus virion. Viruses used in various embodiment may include but are not limited to the Vesicular Stomatitis Virus (VSV) or RV, most preferably the virus used in the various embodiments is an RV virus.

An enhanced immune response is one where the chimeric Rhabdovirus Nfu-antigen fusion protein or an antigen protein incorporated into the glycoprotein of a Rhabdovirus virion is capable of boosting, heightening, prolonging, and/or intensifying the immune response of an inoculated subject to an antigen compared to the immune response of an inoculated subject with a conventional carrier for the antigen.

Protection from Anthrax requires a strong B-cell mediated humoral response directed against Anthrax PA and that by using RV as a carrier for Anthrax PA it is possible to induce such responses. Antigens presented in such a rigid and repetitive organization are sufficient to activate B cells to proliferate and induce IgM production. This has been shown for antigens like VSV glycoprotein, which is highly immunogenic if presented by the viral particle but does not activate B cells as a soluble antigen. The insertion of foreign epitopes into very large DNA viruses or virus-like particles (VLPs) are already shown in different systems: i) hepatitis B core antigen (HBcAg) VLPs containing an epitope of Hepatitis B surface antigen (HBsAg), papillomavirus, or plasmodium falciparum; ii) influenza virus in which epitopes of HIV-1 GP41 or plasmodium yoelli are incorporated into hemagglutinin; iii) HBsAg lipoprotein VLPs displaying epitopes of HIV-1 GP41; iv) hepatitis E virus VLPs expressing an epitope from HSV glycoprotein D. Additionally, a variety of foreign peptides have been chemically coupled to HBcAg and bacteriophage Qβ VLPs. Even though these approaches confirmed that presentation of the foreign epitopes in a highly ordered manner induce strong B-cell responses, they are restricted to relatively short peptides. In contrast, RV based vectors can incorporate whole proteins in its envelope or capsid protein. Stronger humoral responses were detected against these foreign proteins when presented by RV particles compared to the “protein only approach”.

It is expected that foreign antigens presented by recombinant Rhabdovirus, and preferably RV ribonucleoprotein (RNP) or virions, induce potent humoral immune responses, and that RV-based vectors are also excellent to induce strong and protective B-cell responses against various bacterial and viral infections, especially bacterial infections which produce toxins. It is expected that Rhabdovirus mediated, and preferably RV mediated T-helper response(s) and the presentation of whole antigens such as but not limited to Anthrax PA, or Botulinum A Hc50, or others to the immune system in a highly organized manner using the Rhabdovirus and preferably the RV is superior than the use of an antigen alone like recombinant Anthrax PA.

RV is a preferred candidate as a delivery vaccine vector for a variety of antigens, like the anthrax antigens and its variants because the inactivated rabies virus has been used to vaccinate humans since 1978 and thus has a significant history of efficacy and safety. The genetic manipulation of RV is possible for the generation of stable recombinant RVs expressing foreign proteins. The RV genome is highly flexible and allows for the expression of multiple foreign genes or antigens up to 6.5 kb. In addition, foreign proteins can be incorporated into RV virions and/or fused to the RV nucleoprotein. RV N-antigen fusion proteins are incorporated into RV RNPs. Recombinant RV virions and RNPs are easy to purify and highly immunogenic. RV N protein is an excellent B-cell antigen. Killed RV virion-based vaccines can be produced in large quantities and new RV based Anthrax or other RV based vaccines could easily be produced in existing pipelines. Killed RV vaccines are well tolerated and side effects are rare in humans or are generally mild. Recombinant RV virions and RNPs are highly stable ensuring the long-term presentation of antigens like Anthrax PA, Botulinum A Hc50, or other antigens to the immune system. RV induced immune responses are long-lasting. Deactivated (killed) RV virions and RNP have been shown to induce strong humoral responses against the foreign antigens without adjuvant. As a relatively small virus, there are better chances for the lack of significant immune competition between the Anthrax-antigen and RV-specific antigens. RV is a negative-strand RNA virus and its genomes cannot integrate into the host cell genome and thus lacks the oncogenic potential found with viruses that have a DNA phase, such as retroviruses.

Fusion proteins expressed by recombinant viruses permit the virus to carry out all the functions of the virus in the viral life cycle. Various recombinant virus genomes may be constructed in order to determine the location within the genome for the gene encoding the protein for which immunity is sought and which permits the continuation of the viral life cycle. The ability to recover a recombinant virus having the inserted gene in the virus genome shows that the location of the inserted gene does not interfere with the ability of the recombinant virus to carry out all the functions of the virus in the viral life cycle. The fusion protein includes a foreign protein or antigen fused to a carrier protein and the fusion protein is expressed by the recombinant virus. The foreign protein may include any protein for which immunity is sought or against which antibodies can be raised. The foreign protein may be a subunit that is immunogenic and is capable of eliciting antitoxic immunity in the host. Such foreign proteins include but are not limited to bacterial toxins such as anthrax, botulinum and viral diseases. The recombinant virus vector includes the nucleic acid sequence to express the foreign protein. Foreign proteins and genes expressing them refer to proteins not expressed by the carrier vector and genes not found in the carrier vector.

Antigens, or functional mutants thereof, which may be used to the form fusion proteins, such as Nfu-antigen or G-antigen with a portion of the ectodomain removed, may separately include antigens or immunogenic portions such as but not limited to HIV-1 Env epitopes and protein, Hepatitis B surface antigen; toxins such as but not limited to Abrin, Anthrax, Aflatoxins, Botulinum toxin, Cholera toxin, Clostridium perfringens, Conotoxin, Diacetoxyscirpenol toxin, HT-2 toxin, Microcystin (Cyanginosin), Modeccin toxin, Ricin, Saxitoxin, Shiga toxin, Staphylococcus aureus toxins; and Cancer antigens: CA-125: a protein on the surface of ovarian cancer cells; CA 15-3: a high molecular weight protein associated with breast cancer cells; PSA (prostate specific antigen): a protein associated with prostate cancer cells; and Mesothelin: a protein over expressed in primary pancreatic and ovarian cancer cells.

Even though wild-type Anthrax PA itself is very safe, a modified PA antigen like an Nfu-D4 or like D4-Glycoprotein would be advantageous because it is not able to translocate EF and LF in any setting. The carboxyl-terminal end of Anthrax PA is involved in cell receptor binding and Anthrax toxin activity. Further analysis indicated a receptor-binding region within the so-called domain 4 (D4, amino acids 596 to 735) of anthrax PA. Antibodies induced against Anthrax PA D4 are 100% protective in the mouse model, and antibodies directed against the other three domains of PA were also able to protect some mice (20-80%) from Anthrax challenge.

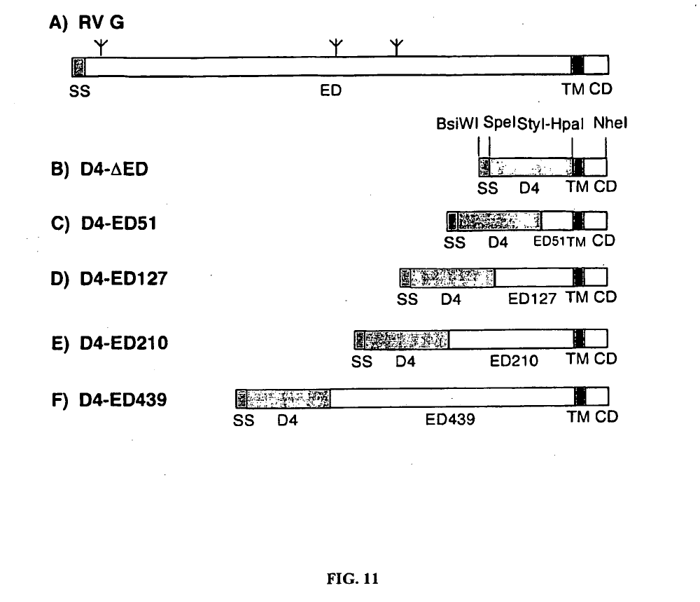

The chimeric polypeptides in some embodiments of compositions can be represented by the formula. Nfu-antigen wherein Nfu represents the nucleoprotein N fragment of a Rhabdovirus like RV or a functional equivalent thereof, and “antigen” represents one or more polypeptides or proteins which are immunogenic. In other embodiments the chimeric polypeptide may be represented by the formula G-antigen, where G represents the Glycoprotein of a Rhabdovirus like RV or its variants and “antigen” represents the component which is one or more polypeptides or proteins which are immunogenic linked to the Rhabdovirus glycoprotein. In some embodiments the chimeric polypeptide RVG-antigen may be modified so that a portion of the ectodomain is deleted and “antigen” represents the component which is one or more polypeptides or proteins which are immunogenic linked to the Rhabdovirus glycoprotein. Non-limiting example include chimeric proteins expressed by the genes show in FIG. 11(B-F). Compositions for therapeutic use may include the polypeptide Nfu-antigen, G-antigen, or combinations of these. Thus the present embodiments encompass the use of the Nfu fragment of a Rhabdovirus, and preferably RV, fused to an antigen, or any other functionally equivalent antigen subunit which would have comparable activities. These polypeptides, and in general these functional equivalents, can be identified by screening methods. The embodiments further encompass the use of the G protein, or functional equivalent thereof which would have comparable activities, for use as a carrier for expression of an antigen, or functional equivalent thereof, and generation of an immune response against the antigen (infra).

Incorporating immunogenic cholera toxin (ctx) B subunit gene into the genome of a Rhabdovirus such as RV may be used to produce a fusion protein. The severe diarrhea that occurs during cholera disease is the result of a host reaction to an extracellular enterotoxin known as cholera toxin. The cholera toxin consists of two different protein subunits which are encoded by the genes ctxA and ctxB. These genes form a single operon called ctx AB (or the ctx operon). A single A subunit and five B subunits make up the complete toxin molecule. It is the A subunit of the cholera toxin which is responsible for the fluid loss characterized by the disease by upsetting the fine control of water and electrolyte balance of the intestinal epithelial cells. The B subunits bind to the host intestinal membrane and perhaps aid the entry of the catalytic A subunit into the host mucosal cells. The B subunit is also immunogenic and is capable of eliciting antitoxic immunity in the host. Cholera toxin is the prototype of a family of bacterial enterotoxins which mediate diarrheal disease and are related in structure, function and immunogenicity. Other members of this family include the heat-labile toxin of E. coli isolated from humans. Common to all of these toxins is an A subunit which mediates ADP-ribosyltransferase activity, resulting in the activation of adenylate cyclase, ultimately leading to death of the target cell. In addition, all of these toxins contain an immunologically dominant B subunit which mediates binding of the holotoxin to the target cell. The B subunit by itself is non-toxic, and immunization with this molecule induces the formation of toxin-neutralizing antibodies.

The bacterium Clostridium botulinum produces the most poisonous biological toxin known. The lethal human dose is a mere 10⁻⁹ mg/kg bodyweight for toxin in the bloodstream. Botulinal toxin blocks nerve transmission to the muscles, resulting in flaccid paralysis. When the toxin reaches airway and respiratory muscles, it results in respiratory failure that can cause death. Incorporating immunogenic BoNTHc into the genome of a Rhabdovirus like RV may be used to produce a fusion protein. For example, genes encoding the BoNT Hc protein or immunogenic portions of the protein may be inserted into the RV system, SPBN-Nfu-BoNT Hc, to express the Nfu-BoNT Hc protein. Alternatively genes encoding the BoNT Hc proteins may be inserted into the glycoprotein of RV system with an intact or partially deleted ectodomain portion, SPBN-BoNT Hc, to express an RVG-BoNT Hc protein. Immunization with these kinds of chimeric glycoproteins can be used to induce the formation of toxin neutralizing antibodies.

N-fusion protein for RV, Vesicular stomatitis virus, Rhabdoviruses or other negative-strand RNA viruses containing a RNP can be constructed by taking an antigen of interest and PCR amplifying it with gene specific primers containing unique restriction enzyme sites (not contained in the gene encoding the antigen of interest). The respective nucleoprotein (N or NP) can be modified in such a way that its stop codon is deleted and a new restriction site introduced which allows cloning of the antigen of interest in frame with the nucleoprotein. Two restriction site 5′ and 3′ of the N-fusion protein can allow functional replacement or the expression of the fusion protein from the recombinant viral genome. For example,

R1-N or NP gene -R2-R3-R4 (virus N or NP protein)

R2-antigen -R3 (antigen of interest, which will be fused to RV N)

R1-N or NP gene-R2-antigen-R3-R4 (final N fusion protein)

R1, R2, R3, R4 are unique restriction sites surrounding the N protein. The unique restriction enzyme sites allow for flexibility in cloning strategies. The choice of a specific site depends on the viral vector and the specific antigen. For example N can be inserted between enzyme site R1 and R2 where site R2 is at the carboxy end of the expressed N protein.

The antigen can be inserted using restriction sites R2 and R3, resulting in a “fusion” gene where, from amino to carboxy, there is a restriction site R1, followed by the N protein sequence, followed by the restriction site R2, followed by the antigen sequence, followed by the remaining restriction sites R3 and R4. The expressed end product is the Nfu-antigen protein. This is one way non-limiting example of making the fusion protein where the N protein and the antigen are in the same reading frame so that both proteins can be made in a single polypeptide. The restriction sites allow flexibility as to what vector and/or antigen can be readily cloned, and into what locations (depending on the restriction sites) of a specific vector.

The location of the restriction sites in the gene encoding the fusion protein to be inserted into the vector can be, for example, a restriction site R1 at the 5′ end of the gene (the amino terminus of the N protein-antigen fusion), the R2 restriction site between/within the sequence encoding the N protein-antigen junction, the restriction sites R3 and R4- at the 3′ end of the gene encoding the antigen (the carboxy terminus of the N protein-antigen fusion). The carboxy terminus of the N protein can be deleted without affecting the ability to form an RNP (supra). Without wishing to be bound by theory, the inclusion of a restriction site between the N protein sequence and the antigen sequence will not harm the expressed fusion protein, as long as the N protein sequence and the antigen sequence are in the same frame.

The insertion of foreign epitopes into viruses or virus-like particles (VLPs) was already shown in a variety of ways. The core antigen of Hepatitis B (HBcAg) in VLPs has been constructed to contain a foreign epitope from Hepatitis B surface antigen (HBsAg), papillomavirus, or Plasmodium falciparum. Another approach uses recombinant influenza virus in which epitopes of HIV-1 gp41 or Plasmodium yoelli are incorporated into the hemagglutinin antigen. Additionally, a variety of foreign peptides have been chemically coupled to HBcAg and bacteriophage Qβ VLPs. Even though these approaches confirmed that presentation of the foreign epitopes in such a manner induces strong B-cell responses, the addition of antigens is in general restricted to relatively short peptides of about 6-10 amino acids. In contrast, RV based vectors can incorporate whole polypeptides/proteins or immunogenic variants of them, (for example the size of GFP is 239 amino acids, PA63 is 568 amino acids, and D4 140 amino acids), in its envelope or into recombinant RNPs. Because RV-derived RNPs are easily produced in the mg range, this approach provides the advantage of producing large amounts of synthesized antigen while allowing for the ability to increase the size, as deemed appropriate, of the foreign antigen to be presented. Of note, another advantage of RV RNPs might be the high stability of RNPs resulting in long-term presentation of the foreign antigen to the immune system.

Previous studies indicate that RV RNP-specific T-helper cells augment the activity of virus neutralization antibody (VNA)-producing B cells. Priming mice with RNPs followed by a boost with killed RV virions resulted in about 10-fold higher B cell responses than RNP priming followed by a boost with RV G that is found in killed RV virions. These results indicate the importance of the physical link of the two RV N and G antigens. The inventors made a similar finding using the recombinant RNPs and the results indicate that RV N does not function as an adjuvant because the addition of wild-type RV RNP was not able to enhance the GFP-specific B cell response when administrated as two separate proteins.

Beside the observed strong B cell response against an antigen presented by RV RNP, the inventors have discovered that the carboxy terminus of RV N seems to be quite flexible and the addition of a whole protein to N did not affect the viral life cycle even though about a third of the total N protein in RNP was the Nfu-GFP fusion protein.

RV-based vectors have been shown to be able to induce potent cellular and humoral immune responses. These vectors however, require the use of a live viral RV vaccine vehicle or killed RV virions containing a foreign glycoprotein. Importantly, these vectors did not utilize an Nfu gene or a modified G gene with portions of the ectodomain removed. Further, because the incorporation of a foreign protein into the rhabdoviral envelope requires surface expression, this approach is only feasible for certain antigens. Moreover, the use of a live viral vaccine vector is only considered when killed vaccines fail to induce immunity. The ability to construct and use recombinant RVs containing RV N-antigen fusion proteins enables them to be expressed and used to protect against other infections which require strong humoral responses.

Killed RV particles expressing chimeric glycoprotein-anthrax PA proteins on their surface may be used as a vaccine carrier to display anthrax PA protein incorporated into the RV virions. These recombinant virions can be purified and used to immunize and challenge mice or other subjects.

Anthrax PA63 is expressed by RV vectors in different cell lines. A recombinant RV expressing recombinant Anthrax PA63 as an extra gene has been constructed to analyze if this protein can efficiently be expressed in eukaryotic cells by RV vectors. As shown in FIG. 2A by immunostaining with an antibody directed against Anthrax PA63, this protein is well expressed with a mainly cytoplasmic location. Genetic modifications can be applied to target PA63 via the ER, Golgi and trans-Golgi to the cell surface to enable incorporation into RV virions.

Rhabdoviruses are able to incorporate a broad variety of foreign viral and cellular glycoproteins into RV virions. Similar to the previously described recombinant RV RNPs, a foreign protein presented in a rigid, multimeric, and high concentration on the surface of RV virions may result in strong B-cell responses. The inventors have determined that HIV-1 Env presented by killed RV virions induces stronger B-cell responses than recombinant HIV-1 Env by itself. HIV-1 gp160 is incorporated into RV virions and induces strong anti-HIV humoral responses. A covalently linked gp140 protein expressing gp120 and the gp41 ecto- and transmembrane domain (ED and TM) fused in frame to the cytoplasmic tail domain (CD) of RV G glycoprotein allows efficient incorporation of this chimeric Env into the RV virions. The use of the killed virions containing the recombinant HIV envelope protein induced strong anti-HIV Env directed immune responses. RV vectors to express these antigens, such as Nfu-antigen or RVG-antigen with a portion of the ectodomain removed, may be constructed. Again, and as seen for the RNP, presentation by RV virions greatly enhanced the observed immune responses compared to the use of “protein only” (data not shown). Soluble RV glycoprotein is a poor B-cell antigen whereas RV G presented on a viral particle is highly immunogenic. The RV N and G proteins contained in the virions may be used to mediate the desired T-cell helper responses to maintain a long-term humoral immune response. This long term humoral immune response against an antigen may be maintained when a chimeric protein, such as an N protein-antigen fusion protein or RVG protein-antigen fusion protein with a modified ectodomain, FIG. 1(I), is expressed by the RV virion. The inventors have determined that priming with RV RNP (not containing RV G) enhances the RV G-directed B-cell responses about 20 fold when boosted with killed virions (containing G) and a similar result is expected with a foreign protein (PA) contained in the RV virions.

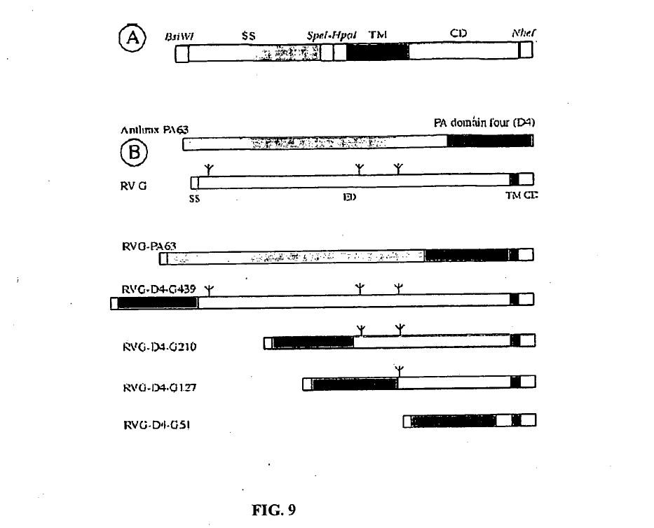

Plasmids encoding Anthrax PA63 or Anthrax PA D4 RV G chimeric proteins may be constructed to express the chimeric glycoprotein-antigen, preferably glycoprotein-anthrax protective antigen or variant, on the surface of the recombinant virions. Plasmids encoding Anthrax PA63 or Anthrax PA D4 RV G chimeric proteins with a portion of the ectodomain removed may be constructed to express the chimeric glycoprotein-antigen, preferably glycoprotein-anthrax protective antigen or variant, on the surface of the recombinant virions. RV incorporates foreign glycoprotein efficiently in their host cell derived viral membrane when fused to the transmembrane domain (TM) and cytoplasmic tail of the RV glycoprotein (G). In addition, the combination of the RV signal sequence (SS) from RV G and the RV transmembrane domain (TM) and cytoplasmic domain (CD) can be used to target a protein via the ER to the cell surface and incorporate such chimeric proteins into RV virions. This was determined for the Hepatitis C(HCV) glycoprotein E1, where sequences encoding the potential ectodomain of HCV E1 were introduced into the expression plasmid pSS-TMT (see FIG. 9A). A similar construction may also be used for Anthrax PA (see FIG. 9B).

Two different construction methods may be used to make such chimeric proteins as illustrated by five different G/PA chimeric proteins (FIG. 9B and FIG. 11). The first construction method can introduce the full-length Anthrax PA63 into pSS-TMT resulting in PA63 with an amino terminal RV G SS and a carboxyterminal TM and CD of RV G. The other construction may use chimeric RV G PA domain four (D4) fusion proteins. To determine if part of the RV G ectodomain (ED) is needed to transport the chimeric RV G/D4 via the ER to the cell surface, five different constructs for this approach based on the inclusion of increasing portions of RV G ED (full-length to about 51 aa or less, or less than a full length ectodomain to about 51 amino acids or less, see FIG. 9B and FIG. 11) were used. While specific amino acids lengths were used as examples of efficacy, longer or shorter amino acid portions of the ED that transport the chimeric RV G/D4 to the cell surface may be used. To make the expression plasmid encoding the chimeric RV G/PA63 protein, PA63 may be amplified by PCR from pGEMPA63 using Vent polymerase (New England Biolabs) and the forward primer PA63-P (SEQ ID. 7) and the reverse primer PA63-M (SEQ ID. 6 and see Table 2: SpeI will not be used for the PA gene because of two internal SpeI restriction sites within PA63, but AvrII has a compatible cohesive end to SpeI). The expected resultant 1.7 kb PCR product (PA63) may be digested with AvrII/HpaI and cloned to the previously SpeI/HpaI digested pSSTMT resulting in pRVGPA63 (FIGS. 9A and B and Table 2).

To construct the chimeric RV G/D4 protein, the sequence encoding D4 may be PCR amplified using the primers D4-P (SEQ. ID. 8) and D4-M (Table 2) (SEQ ID NO:15). The expected resultant PCR product can be digested with AvrII/HpaI and cloned to the previously SpeI/HpaI digested pSSTMT-D4. To introduce the different portions of the RV G ED in frame with D4 (see FIG. 9B, and FIG. 11, Table 2), the forward primers RV-ED439 (SEQ ID NO:16), RVED210 (SEQ ID NO:17), RV-ED127 (SEQ ID NO:18), RV-ED51 (SEQ ID NO:19) and the reverse primers RVG-ED-M (SEQ ID NO:20) may be utilized. The resulting PCR fragments can be digested with HpaI/XbaI and cloned to pSSTMT-D4 previously digested with HpaI/XbaI. The resulting plasmid may be designated pRVG-D4-G439, pRVG-D4-G210, pRV-D4-RVG127, pRVG-D4-G51 (FIG. 9, FIG. 11, Table 2).

These chimeric RV G/PA proteins may be cloned using established techniques. There is a reasonable expectation that the trafficking of theses recombinant proteins to the ER and the transport to the cell surface may work for some of these constructs since recombinant PA63 flanked by an ER translocation signal sequence and the TM and CD of Newcastle Disease Virus (NDV) Hemagglutinin-Neuraminidase (HN) is transported efficiently to the cell surface (Construct is a gift from Drs. Palese and Garcia-Sastre, Mount Sinai School of Medicine, New York). Even though NA is a type-1 glycoprotein, these observations indicate that the proposed transport of PA63 to the cell surface is possible. Further, using a similar methods, it is possible to target HCV E1 to the cell surface. HCV E1 is normally located in the ER membrane. In addition, if the transport of full-length PA63 fails, there is still a good chance that the shorter D4 construct can transported as expected. As indicated above, antibodies directed against Anthrax PA D4 are protective in the mouse model so this shorter D4 version may be sufficient for a vaccine approach. Of note, the inclusion of carboxyterminal RV G fragments of different sizes (see FIGS. 9B and 11), which contain one, two or three of the RV G N-glycosylation sites are likely to support the transport of the chimeric RVG/PA construct. Utilizing different approaches will provide several constructs allowing the surface expression of Anthrax PA or D4 and their incorporation into RV virions.

The carboxyl-terminal end of Anthrax PA is involved in receptor binding and Anthrax toxin activity. Further analysis indicated a receptor-binding region within the so-called domain 4 (D4, amino acids 596 to 735) of Anthrax PA (supra). Antibodies induced against Anthrax PA D4 are 100% protective in the mouse model. Certain regions of the RV G ectodomain might be important to transport PA D4 to the cell surface. To determine which part of the RV G ectodomain (ED) is needed to transport the chimeric RV G/PA63 D4, or other similar chimeric RVG-antigen, via the ER to the cell surface, five different constructs (supra) were utilized for this approach based on the inclusion of increasing portions of RV G ED (full-length to 51 aa, FIGS. 9B and 11). As illustrated in FIG. 1(I) for various antigens, a different number amino acids from the ectodomain may be present in expressed glycoproteins provided they transport the chimeric RVG-antigen to the cell surface. These RVG-antigen fusion proteins may include the full ectodomain, or may include ectodomains with at least one amino acid removed. Although RVG-antigen fusion proteins with ectodomains having at least about 51 amino acids were prepared in working examples, embodiments of RVG-antigen fusion proteins are not limited to ectodomains having 51 amino acids. Using the methods and compositions of this disclosure, RVG-antigen fusion proteins with ectodomains having less than 51 amino acids or greater than 51 amino acid portions of the glycoprotein ED that transport the chimeric RVG-antigen to the cell surface may be made and used.

The incorporation of a foreign glycoprotein into RV virions involves cell surface expression; these experiments will indicate which constructs are surface expressed. Using a transient system based on BSR cells expressing T7 RNA polymerase allows the expression of the chimeric proteins, which are under control of a T7-promoter. Therefore, the respective plasmid encoding the chimeric G/PA protein can be transfected into BSR cells. 48 hours after transfection, cells can be fixed with 4% paraformaldehyde and immunostained using a polyclonal rabbit antibody directed against Anthrax PA and FITC conjugated donkey anti-rabbit and analyzed by fluorescence microscopy. Permeabilized control cells will ensure the expression of the recombinant proteins. This assay can be used for analyzing if the protein is expressed at the surface. The anti-PA rabbit antibody is reactive against PA D4. Internal controls can be included to indicate if the respective chimeric protein is expressed. RV G can serve as a positive control for a protein expressed at the cell surface.

The results shown in FIG. 12 indicate that the PA63 D4 construct is transported to the cell surface if 51 amino acids of the RV G ectodomain are fused in frame to PA63 D4 (D4-E51). Different constructs containing longer or shorter amounts of the RV G ectodomain (ED) may also be used to transport the foreign antigen to the cell surface.

Following identification of the chimeric RV G/antigen proteins, such as RV G/PA, that are surface expressed, the respective genes can be introduced into the attenuated RV expression vector SPBN between the G and L gene. Recombinant RVs can be recovered as described previously. The encapsidation of the genome/anti-genome seems to prevent recombination for Rhabdoviruses and was never detected for any of the inventors recombinant RVs or VSVs.

Sucrose purification of RV virions may be used. For example about 10⁷ BSR cells in a T125 flask may be infected for sixty hours with a MOI of 0.1 with the respective recombinant RV; supernatants can be pre-clarified at 14,000 rpm for 3 minutes in a tabletop centrifuge (Eppendorf Inc.), and spun over 20% sucrose in an SW28 rotor (Beckman Inc.) at 25,000 rpm for one hour. Virion pellets can be resuspended in 1 ml PBS and spun again as indicated above. Viral pellets can be resuspended in 500 μl PBS. Deactivation for immunizations: The sucrose purified recombinant RV virions may be deactivated by incubation with β-Propiolactone (1:1000) overnight at 4° C. followed by another incubation at 37° C. for 30 minutes. This is the standard method use to deactivate rabies vaccines for human use, although other methods may also be used. A Bradford assay can be used to quantify the amount of virions for each virus/preparation. Equal amounts of virions can be used for the immunization. Biochemical characterization of recombinant RV virions can be performed.

Even though surface expression of the chimeric RV G/PA is a good indication of the incorporation into RV virions, the next step is to analyze which proteins are incorporated into RV virions and at what level. Recombinant purified particles were separated by SDS-PAGE and transferred to a nitrocellulose membrane. Western blotting with an Anthrax PA63 specific antibody confirmed incorporation chimeric RV G/PA into the RV virions. Purified RV G and recombinant Anthrax PA can be run in parallel as a standard to quantify the amount of these proteins. Blots may be probed with polyclonal antibodies directed against the Anthrax PA or RV G. Fluorescence analysis may be performed with a Molecular Imager FX (BIO-RAD) with Quantityl (BIO-RAD).

Vaccination and comparison of existing and RV-viron based Anthrax vaccines may be performed as previously detailed for the Nfu-PA63 fusion proteins, except RV virions containing chimeric proteins with antigens like Anthrax PA63, D4, or others instead of RNPs containing Nfu-PA63 or D4 are used. Depending upon the outcome of the construction and characterization of the recombinant viruses different construct can be used to prepare vaccines for further analysis. For example, groups of 5 mice (such as, but not limited to, BALB/c, Swiss Webster, etc.) can be immunized with recombinant virions containing Anthrax PA63 or D4 as indicated in the Table I. Immune responses can be compared to mice immunized with recombinant Anthrax PA.

TABLE 1

| Vaccine | applied dose | comments |
|---|---|---|
| Recombinant Anthrax | 5.0 μg, i.m | Anthrax antigen only (rPA) - |
| | | (protein only) |
| Recombinant virions | | |
| containing PA63/D4 | | |
| Nfu-PA63 virions | 20 μg equal | ~5 μg PA63 (depends on |
| | | incorporation rate) |
| RV virions (wt) | 20 μg | negative control |

Small samples of blood, which can be extracted retro-orbitally from subjects, can be collected after 5 days and weeks 4, 5, 9 and 10 to follow the induced Anthrax PA primary-, and secondary immune responses after immunization. Immunized mice may be euthanized at 10 weeks after the initial immunization, and all blood collected, and spleens removed. The larger amount of blood/serum can be used to perform the cell-based Anthrax toxicity and proliferation assays can be used to analyze the T-helper response for RV G and Anthrax PA. Certain groups of mice can be kept for a longer time (3-6 months) to analyze if the detected anti-PA responses are long-lived which is advantageous for a vaccine.

Assays to analyze Anthrax and RV specific immune responses include the ELISA assay using for example Anthrax PA or RV G; the proliferation assay; an in vitro toxicity testing using mouse macrophages; an in vivo toxicity testing using PA and LF as challenge; and in vivo toxicity testing using Bacillus anthracis.

If the chimeric Nfu-PA and virions expressing PA as vaccines show strong humoral response in mice, such Anthrax vaccine could be moved into a monkey model/Anthrax challenge model.

Depending on the intended mode of administration, the compounds of the present embodiment can be included in various pharmaceutical compositions. The compositions may include a unit dose of the Nfu-antigen fusion proteins such as Nfu-PA63 (SEQ ID NO: 28), an RVG-antigen modified so that a portion of the ectodomain is deleted like RVG-D4-G51, or combinations of these, in combination with a pharmaceutically acceptable carrier and, optionally, can include other medicinal agents, pharmaceutical agents, carriers, adjuvants, diluents, and excipients. “Pharmaceutically acceptable” means a material that is not biologically or otherwise undesirable, i.e., the material can be administered to an individual along with the fusion protein or other composition without causing any undesirable biological effects or interacting in a deleterious manner with any of the other components of the pharmaceutical composition in which it is contained. The pharmaceutical compositions may also be part of diagnostic and pharmaceutical packs and kits comprising one or more containers filled with one or more of the ingredients of the aforementioned compositions.

Examples of physiologically acceptable carriers include saline solutions such as normal saline, Ringer's solution, PBS (phosphate-buffered saline), and generally mixtures of various salts including potassium and phosphate salts with or without sugar additives such as glucose. Suitable excipients are, for example, water, saline, dextrose, glycerol, and ethanol. Nontoxic auxiliary substances, such as wetting agents, buffers, or emulsifiers may also be added to the composition. In one embodiment of the embodiment, adjuvants are not required for immunization.

Parenteral administration, if used, is generally characterized by injection. Sterile injectables can be prepared in conventional forms, either as liquid solutions or suspensions, solid forms suitable for solution or suspension in liquid prior to injection, or as emulsions.

For each recipient, the total vaccine amount to be administered can be deduced from protocols for immunization with other vaccines. The exact amount of Nfu-antigen or killed virions having glycoprotein-antigen compositions will vary from subject to subject, depending on the species, age, weight and general condition of the subject, the particular fusion protein used, its mode of administration, and the like. Generally, dosage will approximate that which is typical for the administration of other vaccines.

The subject or recipient of the Nfu-antigen or virion or virions having a glycoprotein-antigen composition may be is a mammal, e.g., a mouse, cat, dog, horse, cow, pig, sheep, goat, or human. Although human use is preferred, veterinary use and testing of the embodiment is also feasible. The vaccine is administered as a sterile composition. The fusion proteins and other compositions of the embodiment can be administered by any suitable means, e.g., parenterally (subcutaneously, intramuscularly, or intraperitoneally), intravenously, or orally. An appropriate evaluation of the time and method for delivery of the vaccine is well within the skill of the clinician.

The timing of administration of the vaccine and the number of doses for immunization can be determined from standard vaccine administration protocols by one of ordinary skill in the art employing no more than routine experimentation (see, e.g., Product Information, Physician's Desk Reference (1996)).

The recombinant virions of the present invention are useful for producing an Nfu-antigenic polypeptide(s), for example the Nfu-D4, or subunits thereof, which reacts immunologically with a biological sample from a patient, such as, but not limited to, serum, containing anthrax D4 antibodies. Embodiments also encompasses antibodies raised against Nfu-anti genic and/or RVG-antigen with modified ectodomain polypeptides, which are useful in immunoassays to detect the presence of these viral chimeric antigens, in biological samples. Design of the immunoassays is subject to a great deal of variation, and many formats are known in the art. The immunoassay will utilize at least one viral epitope that is derived, for example, from Nfu-antigen. In one embodiment, the immunoassay uses a combination of viral epitopes derived from Nfu-antigen. These epitopes may be for example the PA63 and D4 antigens of Anthrax PA. An immunoassay may use, for example, a monoclonal antibody directed towards a viral epitope(s), a combination of monoclonal antibodies directed towards epitopes of one viral antigen, monoclonal antibodies directed towards epitopes of different viral antigens, polyclonal antibodies directed towards the same viral antigen, or polyclonal antibodies directed towards different viral antigens.

Protocols may be based, for example, upon competition, or direct reaction, or sandwich type assays (infra). Protocols may also, for example, use solid supports, or may be by immunoprecipitation. Most assays involve the use of labeled antibody or polypeptide; the labels may be, for example, enzymatic, fluorescent, chemiluminescent, radioactive, or dye molecules. Assays that amplify the signals from the probe are also known; examples of which are assays which utilize biotin and avidin, and enzyme-labeled and mediated immunoassays, such as ELISA assays

Various aspects of the present embodiment will be illustrated with reference to the following non-limiting examples.

### EXAMPLE 1

This example describes Plasmid construction and generation of recombinant viruses. The plasmids pBNSP, pSPBN and pSPBN-333 have been described previously. To construct a recombinant RV that allows the cloning of a foreign gene in frame to the 3′ end of RV N, pBNSP was digested with PstI and NheI and the ˜1.6 kb fragment was replaced with a ˜1.6 kb PCR-amplified from pBNSP using Vent® polymerase (Biolabs Inc.) and the primers RP201 5′-AAAACGTACGTGAGTCACTCGAATATGTCTTG-3′ (SEQ ID NO: 1) and RP64 5′-TGTGCTGCAAGGCGATTAAG-3′ (SEQ ID NO: 2). The resulting plasmid was designated pBNSP-Nfu and used to introduce the GFP gene. The EGFP sequence was PCR amplified by PCR using the primers RP199 5′TTTCGTACGGTGAGCAAGGGCGAGGAGCTG-3′ (SEQ ID NO: 3) and RP200 5′-AAAGCTAGCTTACTTGTACAGCTCGTCCATG-3′ (SEQ ID NO: 4) and cloned in frame with the RV N gene utilizing the BsiWI and NheI restriction sites. The construct was named pBNSP-Nfu-GFP. To clone the sequence encoding the Nfu-GFP fusion protein as an additional gene between the RV G and L genes, this Nfu-GFP sequence was PCR amplified by PCR from pBNSP-Nfu-GFP using the primers RP266 5′-TTTGGTACCACAATGGATGCCGACAAGATTG-3′ (SEQ ID NO: 5) and RP200. The ˜2 kb PCR product was digested with Acc65I and NheI and cloned into pSPBN-333 digested with BsiWI and NheI. The resulting plasmid was designated pSPBN-Nfu-GFP. The recombinant viruses were recovered as described previously and designated BNSP-Nfu and BNSP-Nfu-GFP.

Sucrose purification of RV virions. BSR cells (a BHK clone) (1.25×10⁷) in a T150 flask were infected for 72 hours at a multiplicity of infection (MOI) of 1 with SPBN-Nfu-GFP virus, or SPBN virus; the supernatants were preclarified at 1400 rpm for 5 minutes and spun over 20% sucrose in an SW27 rotor (Beckman, Inc) at 21,000 rpm for 0.1 hour at 4° C. Virion pellets were resuspended in 200 μl phosphate-buffered saline (PBS.)

RV RNP. BSR cells in six T150 (150 cm²) tissue culture flasks were infected with SPBN virus, or SPBN-Nfu-GFP virus at an MOI of 0.01 for 72 hours. The infected cells were then washed once in PBS (pH7.4), scraped from the flask and resuspended in 4 ml ice-cold distilled water containing aprotinin (50 mg/ml). The cells were homogenized at 20,500 rpm and cell debris was pelleted by centrifugation at 3000 rpm. Supernatants were removed and saved and then pellet was resuspended in 3.5 ml ice cold distilled water containing aprotinin (50 mg/ml) and homogenized again. Cell debris was again pelleted and supernatants were combined and clarified by centrifuging in SW-27 rotor (Beckman, Inc) for 10 minutes at 10,000 rpm 4° C. The supernatant was collected and 100 μl of 10×PBS was added before being combined with 3 g cesium chloride and centrifuged overnight in SW-55 rotor (Beckman, Inc) at 36,000 rpm 19° C. The RNP bands were collected using a 23-gauge needle. Protein concentration was determined using the BCA Protein Determination kit (Pierce).

Western blotting (i) virions. Proteins from lysed virions were separated in 10% SDS/PAGE gel and transferred to a PVDF-Plus membrane (Osmonics, Minnetonka, Minn.). Blots were blocked for one hour in 5% dry milk powder in PBS (pH 7.4), then washed three times using Western blot wash solution (WBWS, [0.1% PBS-Tween-20]) and incubated with a monoclonal mouse α-GFP antibody (1:1000) (Sigma), or a polyclonal rabbit α-RV RNP antibody (1:2000) overnight at 4° C. Blots were then washed three times with WBWS and secondary goat α-mouse IgG (1:10,000) or goat α-rabbit IgG (1:25,000) (Jackson ImmunoResearch Laboratories) conjugated to HRP were added. Blots were then incubated for one hour at room temperature and after three 10-minute washes with WBWS and one wash with PBS (pH 7.4), chemiluminescence was performed as instructed by the manufacturer (Perkin Elmer Life Sciences). Blots were read on X OMAT AR film (Kodak).

(ii) RNP fractions. RV RNPs were purified from BSR cells infected with SPBN, or SPBN-Nfu-GFP as described above and collected from the bottom of the centrifuge tube in 250 μl fractions and dialyzed against PBS two times overnight at 4° C. Proteins from purified ribonucleoproteins were separated an a 10% SDS/PAGE gel and transferred to a PVDF-Plus membrane. Blots were blocked for one hour in 5% dry milk powder in PBS (pH 7.4), then washed three times using (WBWS), and incubated with a monoclonal mouse α-GFP antibody (1:1000) (Sigma), or a polyclonal rabbit RV α-RNP antibody (1:2000) overnight at 4° C. (site). Blots were then washed three times with WBWS. Secondary goat α-mouse IgG (1:10,000) or goat α-rabbit IgG (1:25,000) conjugated to HRP was added, and blots were incubated for one hour at room temperature. After three 10-minute washes with WBWS and one wash with PBS (pH 7.4), chemiluminescence was performed as listed previously.

One-Step growth curve. BSR cells were plated in 60 mm dishes and 16 hours later infected at an MOI of 5 with SPBN virus or SPBN-Nfu-GFP virus. After incubation at 37° C. for 1 hour, the inocula were removed and cells were washed four times with PBS to remove any unabsorbed virus. Three milliliters of complete medium was added back and 100 μL of tissue culture supernatants were removed at the indicated time points after infection. Virus aliquots were titered in duplicate on BSR cells.

CD4 Depletion. Six C57BL/6J female mice (Jackson Laboratories) aged 6-8 weeks were depleted of CD4 cells prior to immunization by treating mice with GK1.5 ascites. Mice were injected i.p. with 40 μl of GK1.5 mouse ascites diluted in 160 μl PBS on days −3, −1, 11 and with 12 μl GK1.5 ascites diluted in 198 μl PBS on days 6 and 18 post immunization. CD4 depletion was analyzed by flow cytometry of depleted and compared with non-depleted splenocytes on the day of immunization.

Immunization. Three groups of 5 C57BL/6J female mice aged 6-8 weeks were immunized i.m. with 5 μg Nfu-GFP (SEQ ID NO: 30), 5 μg wild-type RNP plus 2 μg recombinant GFP (BD Biosciences), or 2 μg recombinant GFP only. All groups of mice were boosted with same amounts as initial priming two and five weeks after primary immunization. Mice were bled retro-orbitally on days indicated and serums were analyzed by ELISA for RNP or GFP-specific antibodies as described below.

Two groups of 5 C57BL/6J female mice (Jackson Laboratories) aged 6-8 weeks (one group CD4 depleted) were immunized i.m. with 10 μg Nfu-GFP (SEQ ID NO:30) and given boosts on day 12 of equivalent amounts. All mice were bled retro-orbitally as indicated and analyzed by ELISA for RNP, or GFP-specific antibodies as described below.

ELISA of anti-RV RNP and anti-GFP antibody titers. RV RNP and GFP-specific antibodies present in mice after immunization were analyzed by enzyme-linked immunoosorbent assays (ELISA) using 5 μg recombinant GFP (BD Bioscience), or 10 μg RV RNP diluted in 10 ml coating buffer (50 mM Na₂CO₃, pH 9.6) as the trapping antigen, 96-well plates (Maxisorp plates, Nunc) were coated with 100 μl in each well and incubated overnight at 4° C. Plates were blocked by incubating for 30 minutes at room temperature (RT) using 5% milk powder in PBS followed by three washes in 0.1% PBS-tween. Serum dilutions were prepared and added to wells as shown in the respective figures and incubated for 1 hour at RT followed by three washes again in 0.1% PBS-tween. Secondary antibody, goat anti-mouse IgG HRP (1:5000) (Jackson ImmunoResearch Laboratories), or goat anti-mouse IgM HRP (1:5000) (Sigma) was added and incubated for 30 minutes at RT followed by three washes in 0.1% PBS-Tween.

O-Phenylendiaminedihydrochloride (OPD) (Sigma) was used to develop HRP-conjugated antibodies for 15 minutes and stopped by adding 3M H₂SO₄. Plates were read at 490 nm using a DuPont kinetic microplate reader.

### EXAMPLE 2

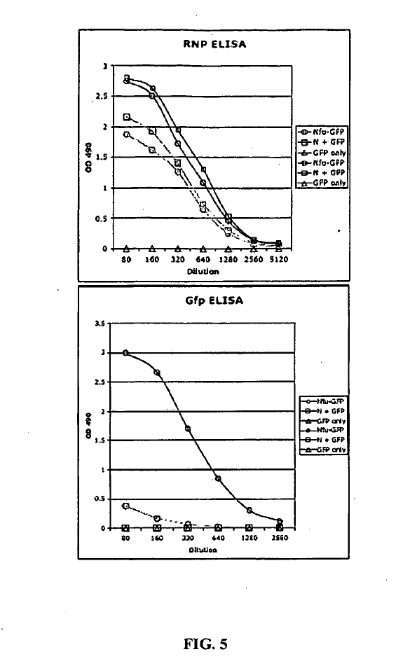

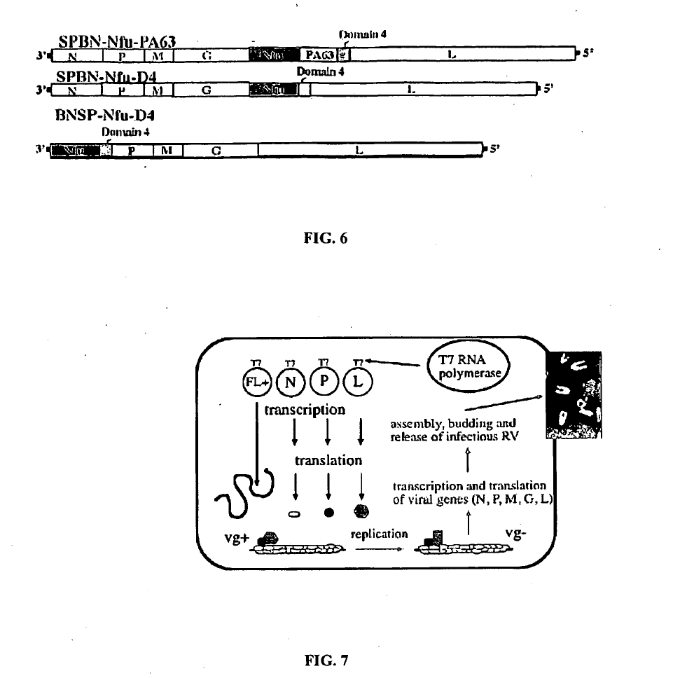

Construction of recombinant RV expressing RV-N Anthrax-PA fusion proteins. FIG. 6 shows the three different RVs expressing Anthrax PA63 (SPBN-Nfu-PA63) or the domain 4 of Anthrax PA63 (D4) (SPBN-Nfu-D4) as an additional gene. BNSP-Nfu-D4 is a recombinant RV where the RV N protein is replaced by the RV-Nfu-D4 protein.

Whereas Anthrax PA is secreted from Bacillus anthracis as an 83-kDa protein, furin protease cleaves PA, releasing a 20-kDa fragment. The 63-kDa portion (PA63) of the cleavage product forms a heptamer, which binds the toxins LF and EF. This complex enters via receptor mediated endocytosis where LF and EF exert their toxic effects. Antibodies directed against Anthrax PA are able to protect against Anthrax infection. RNPs containing RV N-fusion proteins are excellent B-cell antigens. The fusion protein of PA63 and PA63 D4 to RV N expressed by recombinant RNPs containing Anthrax PA or a subunit of PA can be used to create Anthrax vaccines.

For a RV RNP based Anthrax vaccine three different recombinant RVs (SPBN-Nfu-PA63, SPBNNfu-D4, and BSPP-Nfu-D4, see FIG. 6) may be constructed. To create the two RVs expressing the Nfu-PA63 (SEQ ID NO: 27) or Nfu-D4 as an extra gene from RV, SPBN-Nfu-GFP can be digested with BsiWI/NheI. The genes encoding Anthrax PA63 or D4 can be amplified by PCR from pGEM-PA63 (42) using Vent polymerase (New England Biolabs) and the reverse primers PA63-Nfu-M (5′-AAAGCTAGCTTATCCTATCTCATAGCCCTTC-3′, NheI site in bold; (SEQ ID NO: 21) and the forward primer PA63-Nfu-P (5′-TTTCGTACGACAATGTCCACAAGTGCTGGACCTAC-3′, BsiWI in bold; SEQ ID. 22) in the case of PA63 or D4-Nfu-P (5′-TTTCGTACGGACAAGCGTTTCCATTATGATA-3′, BsiWI in bold; SEQ ID. 23) in the case of Anthrax PA D4. The 1.7 kb (PA63) or 0.43 kb (D4) PCR fragments can be digested with BsiWI/NheI and cloned to SPBN-Nfu-GFP. To create an RV vector where we replace RV N with RV Nfu-D4, the 0.43 kb D4 fragment can be digested with and cloned to the previously digested BNSP-Nfu. Sequences of the fusion proteins and the flanking sequences will be confirmed by sequencing for all three constructs.

Based on studies with a recombinant RV expressing a Nfu-GFP protein (SEQ ID NO: 30), it is expected that it will be possible to recover an RV expressing the RV Nfu-Anthrax PA63 or D4 fusion proteins. It is expected that these proteins will be incorporated into the recombinant RNPs as seen for GFP. Because PA63 is about double the size of GFP, this protein might be incorporated in a lower amount, the size of D4 is only half of that of GFP and therefore Anthrax D4 should be incorporated at least as efficiently as GFP.

### EXAMPLE 3

This example describes a Rabies virus recovery system as illustrated in FIG. 7. BSR cells (a BHK cell clone) stably expressing bacteriophage T7 RNA polymerase are transfected with four different plasmids (circles), three plasmids encoding the RV virus viral proteins [nucleoprotein (N), phosphoprotein (P), polymerase (L)], and one encoding the anti-genomic RNA of the respective recombinant RV. All plasmids contain the promoter for the T7 RNA polymerase. A successful recovery utilized the encapsidation of the anti-genomic RNA into the nucleoprotein building the ribonucleoprotein (RNP, vg+ see FIG. 7) and the subsequent replication by the RV polymerase complex (P and L protein) to the genomic RNP (vg−). The genomic RNP serves as a template for expression of all viral protein N, P, M (matrix), G (glycoprotein) L, and the assembly, budding and release of infectious RV from the cell membrane. The newest vg+ contains a hammer head ribozyme at the 3′ end.

Recovery of recombinant RV from cDNA. The study of the non-segmented, negative-stranded RNA viruses has been limited by an inability to manipulate the viruses using recombinant DNA technology. The inventors have discovered the generation of a non-segmented negative-stranded RNA virus, RV, entirely from cDNA. The approach involved intracellular expression of antigenomic RNA in cells also expressing the viral proteins used for formation of an active RNP complex, namely, the nucleoprotein (N), the phosphoprotein (P), and the viral polymerase (L) (FIG. 7). This method avoids problems of antisense RNA that are encountered when expressing the non-encapsidated negative-strand genomic RNAs, and positive stranded mRNA. This method was also successful in the recovery of another rhabdovirus, VSV, and paramyxoviruses. The initial recovery frequency of recombinant RV from cDNA was very low, occurring in only a very small number of the transfected cells. This was probably due to interference of RV and the recombinant vaccinia virus expressing T7 RNA polymerase that was used to express the RV full-length RNA and the RV proteins. Using a BSR cell-line stably expressing T7 RNA polymerase instead of recombinant vaccinia virus, it is possible to increase recovery frequencies to as high as about 100%.

Using the RV recovery system, it was possible to recover more then 50 different RVs expressing genes such as chloramphenicol acetyltransferase (CAT), luciferase, β-galactosidase, HIV-1 envelope proteins, HIV-1 Gag, SIVmac239 Env, and Hepatitis C (HCV) E2, HCV E1E2p7. Where certain foreign genes, such as Anthrax PA63 that, contain a sequence similar to the short transcription Stop/Start sequence within the RV genome that would result in an internal termination of transcription, such sequences can be eliminated by site-directed mutagenesis.

To analyze whether the recombinant RVs are expressing PA63 or D4 BSR can be infected with an MOI of 0.1 for 48 hours, fixed, and immuno-stained with an antibody directed against PA63 or RV N. The result will indicate if recombinant RVs are expressing Anthrax PA/D4. To determine whether the recombinant viruses incorporate the recombinant N-fusion proteins (Nfu-PA63 or Nfu-D4) into the RNP, BSR cells can be infected with an MOI 5. About forty-eight hours later, supernatants from the infected cells can be harvested, cells and cell fragments removed and the virions purified over 20% sucrose. Virion proteins may be separated by SDS-PAGE, transferred to a PVDF-Plus membrane, and analyzed by Western blotting with antibodies directed against Anthrax PA, or RV RNP. The ratio of RV N to RV Nfu-PA will indicate the ratio of incorporation. Well-characterized recombinant RV Nfu-protein, like RV SPBN-Nfu-GFP can be used as a control throughout these experiments.

### EXAMPLE 4

This prophetic example describes the isolation and preparation of RV RNPs for immunizations.

Methods: BSR Cells in 3 T125 (125 cm²) tissue culture flasks can be infected with an MOI of 0.1 with the respective recombinant RV expressing an Nfu-PA for 48 hours. Infected cells will be scraped from the tissue culture flasks, washed once with PBS and re-suspended in 5 ml of ice-cold distilled water containing aprotinin (1000 units). After homogenizing the cells, cell debris will be removed by centrifugation for 20 minutes at 1000 g. Supernatant will be saved, cell pellet re-suspended as indicated above, centrifugated and both supernatants combined. After an addition clarification step by centrifugation for 10 minutes at 12,000 g, 6 grams CsCl can be added and volume adjusted to 15 ml. After centrifugation for 18 hours at 150,000 g at 40 C, RNPs are expected to form a sharp band at buoyant density of 1.31 g/cm³. The RNP band can be collected using a 23-gauge needle and dialyzed against PBS. Protein concentration may be determined by a standard Bradford assay.

Where a lower amount of RNP is derived from SPBN-Nfu-GFP compared to wild-type, suggesting that the recombinant SPBN-Nfu-GFP replicated at reduced rate and therefore production of viral proteins was reduced, one step growth curves and immunostaining can be used to determine when production of RV N/Nfu is highest.

### EXAMPLE 5

This prophetic example describes immunizations and studies of the Anthrax PA-specific immune responses.

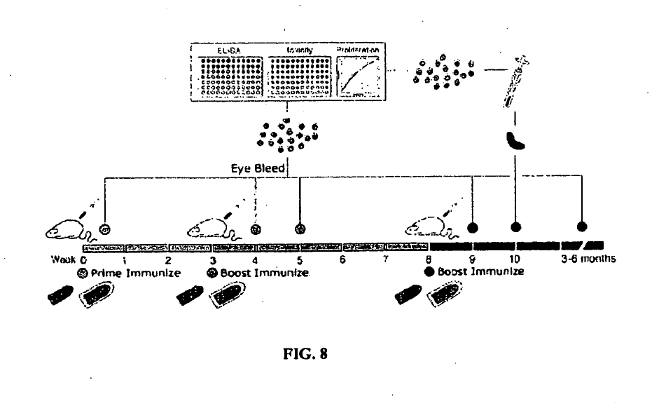

As outlined in FIG. 8, blood can be collected at 5 or 6 (for long-term) different time-points for a detailed study of the induced anti-Anthrax PA responses during immunization. The induction RV N or GFP-specific antibody responses utilize an initial priming with the Nfu-GFP RNPs followed by a second inoculation with the same antigen and it may be expected that Nfu-PA or other fusion proteins such as Nfu-antigen with utilize similar priming. An ELISA assay based on recombinant Anthrax PA can be used to initially follow the B-cell responses. PA directed antibodies may be further characterized by proliferation assays, and in vitro and in vivo toxicity testing and used to prioritize the selection and development of new and improved vaccine vehicles in a monkey model.

Comparison of existing and RV-based Anthrax vaccines to Nfu-PA compositions can be made by initially immunizing for example, about 5 groups of 5 mice with recombinant RNPs containing Anthrax PA63 or D4 as indicated below (see also FIG. 8). Immune responses may be compared to mice immunized with the human Anthrax vaccine AVA BioThrax™, recombinant Anthrax PA, or a mixture of rPA and RNP-wt. Including the mixture of rPA and wild-type RNP can be used to determine if RV RNP has an adjuvant effect and determine if RV N has to be linked to PA to provide the required T-helper responses. A group of 5 mice immunized with RNP derived from wild-type RV (RNP-wt) serves as a negative control.

| Vaccine | applied dose | comments |
|---|---|---|
| Recombinant Anthrax | 5.0 μg, i.m | Anthrax antigen only (rPA) - |
| | | (protein only) |
| Recombinant virions | | |
| containing PA63/D4 | | |
| Nfu-PA63 virions | 20 μg equal | ~5 μg PA63 (depends on |
| | | incorporation rate) |
| RV virions (wt) | 20 μg | negative control |

Small samples of blood, can be extracted retro-orbitally will be collected after 5 days and weeks 4, 5, 9 and 10 to follow the induced Anthrax PA primary-, and secondary immune responses after immunization. Sacrifice the immunized mice 10 weeks after the initial immunization, all blood will be collected, and spleens removed. The larger amount of blood/serum will enable cell-based Anthrax toxicity tests to be performed. Proliferation assays to analyze the T helper response may also be performed. It is expected that RV N protein, when fused to the Anthrax PA, will provide T-help. Such responses should be detected by proliferation assay using Anthrax PA or RV N proteins. In addition and as illustrated in FIG. 8, certain groups of mice may be kept for a longer time (3-6 months) to analyze if the detected anti-PA responses are long-lived, which is advantageous for a vaccine.

Five different assays may be used to study the anti-Anthrax immune response. ELISA assay for anti-PA or anti RV N antibodies, one of the fastest screening methods to follow the anti-Anthrax or anti-RV nucleocapsid responses is an ELISA assay. For ELISA only small serum samples are needed, which can be collected by retro-orbitally extraction from immunized mice. Proliferation assay for Anthrax PA RV N indicates CD4 T-helper responses utilized for long-term protection. In vitro toxicity testing indicates if the detected immune responses neutralize Anthrax PA in a cell based assay. In vivo toxicity testing using PA and LF as challenge in small animal model is used to analyze protection against Anthrax toxins without the need to use B. anthracis. In vivo toxicity testing using Bacillus anthracis in small animal model can be used to analyze protection against Anthrax bacteria/spores.

### EXAMPLE 6

This prophetic example describes an ELISA assay for anti-PA or anti RV N antibodies. RV N protein may be isolated from purified RNPs by well-established methods (see RNP isolation). Recombinant Anthrax PA can be purchased from List Biological Laboratories Inc., Campbell, Calif., USA. Anthrax PA or RV N is resuspended in ELISA plate coating buffer (50 mM Na₂CO₃, pH 9.6) at a concentration of 200 ng/ml and plated in 96-well ELISA MaxiSorp plates (Nunc Inc.) with 100 μl in each well. After overnight incubation at 40° C., plates can be washed three times (PBS pH 7.4, 0.1% Tween-20), blocked with blocking buffer (PBS, pH 7.4, 5% dry milk powder) for 30 minutes at room temperature, and incubated with serial dilutions of sera for 1 hour. Plates can be washed three times followed by the addition of horseradish peroxidase-conjugated (HRP) goat anti-mouse-IgG (H+L) secondary antibody (1:5000, Jackson ImmunoResearch Laboratories). After about 30-minute incubation at 37° C., plates can be washed three times and 200 μl OPD-substrate (o-phenylenediamine dihydrochloride, Sigma) is added to each well. The reaction is stopped by the addition of 50 μl of 3 M H₂SO₄ per well. Optical density is determined at 490 nm. Monoclonal antibodies directed against Anthrax PA or RV N protein will serve as controls.

ELISA is used to detect RV N-specific humoral responses in serum of immunized animals is a standard method in the laboratory. An established anti-PA ELISA can be established using method developed for assays with HIV-1 Env, Gag and GFP. Usually, the amount of antigen used for a specific ELISA assay has to be titered. In addition, different coating buffers can be used. Using both monoclonal and polyclonal anti-PA directed antibodies will facilitate the set up this additional anti-Anthrax ELISA.

### EXAMPLE 7

This prophetic example describes the RV N and PA63 specific Proliferation assay. Prepared RV-based killed Anthrax vaccines of the present embodiment may be compared to the efficacy to other approaches like the human AVA vaccine. T-helper cell responses are the most important factor for long-term IgG responses. Without wishing to be bound by theory, it is possible that the observed strong humoral responses detected against GFP presented as a fusion protein with RV N was induced due to a strong RV N-dependent T-helper response. CD4+ T-helper responses are important for both differentiation and the creation of memory B-cells.

A strong CD4+ T cell helper response should be elicited by a vaccine candidate in order to obtain strong B cell responses. Antigen-specific T-cell proliferation is a major technique for assessing the functional capacity of CD4+ lymphocytes to respond to various stimuli. Spleens from euthanized immunized subjects, like mice, may be removed at different time points (ten weeks, three and six months) and single cell suspensions prepared. Red blood cells are lysed with ACK lysing buffer (BioSource) and cells washed twice in RPMI-10 supplemented with 10% FBS. Splenocytes are seeded in 96-well microtiter flat bottom plates (106 cells/well) in triplicate and stimulated with Anthrax PA or RV N protein at experimentally determined concentrations. After a three-day incubation period, cells can be labeled with [3H]thymidine (1 μCi/well) for an additional 12-18 hours. The plates are harvested and [3H]thymidine incorporation measured by liquid scintillation counting. To ensure cells are healthy during the assay, triplicate wells of splenocytes will also be incubated with PHA (10 μg/ml) as a positive control. Stimulation index will be calculated as follows: Stimulation index=(experimental count−spontaneous count)/spontaneous count). Adjustment of the amount of antigen used for stimulation and time for stimulation can be modified to get reliable and reproducible results in this assay system.

### EXAMPLE 8

This prophetic example describes the in vitro protection assay. Whereas the initial screening by an ELISA assay for anti PA antibodies is the fastest method to follow the humoral responses in immunized mice, the reduction of cytotoxicity on RAW 264.7 mouse macrophages or CHO-CL6 is the better system to analyze if these antibodies are protective. This system is based on the treatment of cells with a lethal combination of PA plus LF. Preincubation of PA with the sera of immunized mice will indicate the potency of this approach to reduce cytotoxicity. PA neutralization assays can be performed with both RAW264.7 (mouse macrophage) and CHO-CL6 cell lines. RAW264.7 cells (mouse macrophage cell line): Serial dilutions of sera from immunized mice are incubated with 100 ng Anthrax PA for one hour at room temperature (RT) to allow neutralization, followed by addition of 500 ng/ml LF. The antiserum-toxin mixture is added to RAW264.7 cells are plated in 96-well plates at a density of 30,000-35,000 cells/well the day before. Each tested dilution can be made in triplicate wells. Cells are incubated at 37° C. for 4 h and cell viability is determined by the addition of 3-[4,5-dimethylthiazol-2-yl]-2,5-diphenyltetrazolium bromide (MTT). Viable cells convert the MTT to an insoluble blue pigment, which is measured. Results may be plotted and analyzed with Prism software (GraphPad Software Inc., San Diego) as the percentage viability of control wells containing LF without PA. CHO-CL6 cytotoxicity assays are performed similarly to RAW264.7 macrophages, with two exceptions: 1) the cells are plated at a lower density of 15,000 cells/well, because after the addition of the toxins (50 ng/ml FP59), cells will be incubated 48 h prior to MTT addition; 2) FP59, a recombinant toxin consisting of the N-terminal PA-binding portion of LF fused to the ADP-ribosylation domain of Pseudomonas exotoxin A will be used in place of LF because CHO cells are not lysed by lethal toxin. EC₅₀ values may be determined for three independent assays as above. Neutralization by rabbit polyclonal anti-PA antibody 5308 will serve as positive control and sera from wild-type RV RNP immunized mice as a negative control for both assays.

Two challenge assays may be used in mice. The first is based on the challenge with PA and LF, the second uses infectious Anthrax spores. In vivo protection experiment. The experiments listed above will indicate the best immunization methods regarding the antigen used and immunization timing. Using this immunization schedule, groups of ten immunized BALB/c and a control group of ten mice can be inoculated i.v (tail vein injection) of a mixed combination of purified PA and LF proteins (60 μg of PA and 30 μg of LF per mouse in PBS); this equals five 50% lethal doses (LD50). Where it is determined that different preparations of PA and LF result in different LD50, new preparations can be made and re-tested for their LD50 in BALBs nice.

### EXAMPLE 9

This prophetic example describes the nucleotide sequence of the mouse PRPa (prion protein) and sequence representing the protease resistant core within the mouse PrP molecule (AA 90-230) for cloning in frame to the 3′ end of the RV N, as described previously (supra) for generation of a recombinant Nfu-prion fusion construct. The primer that can be used to fuse these sequences, the prion protein or the protease resistant core of the prion protein, in frame with RV N are listed below. Based on studies with a recombinant RV expressing a Nfu-GFP protein (SEQ ID NO: 30) (supra), it is expected that it will be possible to recover an RV expressing the RV N prion fusion (or the protease resistant core of the prion protein fusion) proteins as an extra gene. It is expected that these proteins will be incorporated into the recombinant RNPs as seen for GFP. Because the prion protein is a relatively large protein, this protein might be incorporated in a lower amount. If this is the case the smaller protease resistant core of the prion protein should be incorporated at least as efficiently as GFP.

PRPa

(SEQ ID NO: 9)

ATGGCGAACCTTGGCTACTGGCTGCTGGCCCTCTTTGTGACTATGTGGAC
TGATGTCGGCCTCTGCAAAAAGCGGCCAAAGCCTGGAGGGTGGAACACCG
GTGGAAGCCGGTATCCCGGGCAGGGAAGCCCTGGAGGCAACCGTTACCCA
CCTCAGGGTGGCACCTGGGGGCAGCCCCACGGTGGTGGCTGGGGACAACC
CCATGGGGGCAGCTGGGGACAACCTCATGGTGGTAGTTGGGGTCAGCCCC
ATGGCGGTGGATGGGGCCAAGGAGGGGGTACCCATAATCAGTGGAACAAG
CCCAGCAAACCAAAAACCAACCTCAAGCATGTGGCAGGGGCTGCGGCAGC
TGGGGCAGTAGTGGGGGGCCTTGGTGGCTACATGCTGGGGAGCGCCATGA
GCAGGCCCATGATCCATTTTGGCAACGACTGGGAGGACCGCTACTACCGT
GAAAACATGTACCGCTACCCTAACCAAGTGTACTACAGGCCAGTGGATCA
GTACAGCAACCAGAACAACTTCGTGCACGACTGCGTCAATATCACCATCA
AGCAGCACACGGTCACCACCACCACCAAGGGGGAGAACTTCACCGAGACC
GATGTGAAGATGATGGAGCGCGTGGTGGAGCAGATGTGCGTCACCCAGTA
CCAGAAGGAGTCCCAGGCCTATTACGACGGGAGAAGATCCAGCAGCACCG
TGCTTTTCTCCTCCCCTCCTGTCATCCTCCTCATCTCCTTCCTCATCTTC
CTGATCGTGGGATGA
PRPa (AA 90-230)

(SEQ ID NO: 10)

CAAGGAGGGGGTACCCATAATCAGTGGAACAAGCCCAGCAAACCAAAAAC
CAACCTCAAGCATGTGGCAGGGGCTGCGGCAGCTGGGGCAGTAGTGGGGG
GCCTTGGTGGCTACATGCTGGGGAGCGCCATGAGCAGGCCCATGATCCAT
TTTGGCAACGACTGGGAGGACCGCTACTACCGTGAAAACATGTACCGCTA
CCCTAACCAAGTGTACTACAGGCCAGTGGATCAGTACAGCAACCAGAACA
ACTTCGTGCACGACTGCGTCAATATCACCATCAAGCAGCACACGGTCACC
ACCACCACCAAGGGGGAGAACTTCACCGAGACCGATGTGAAGATGATGGA
GCGCGTGGTGGAGCAGATGTGCGTCACCCAGTACCAGAAGGAGTCCCAGG
CCTATTACGACGGGAGAAGATCC
Primers to construct SPBN-PRPa:
PRPa1 (+) (42 mer) (with BsiWI site)

(SEQ ID NO: 11)

AGA TgT ATA CgT ACg AAg ATG GCG AAC CTT GGC TAC
TGG CTG
PRPa2 (−) (43 Mer) (with NheI site)

(SEQ ID NO: 12)

TCT ATC AgC TAg CTA gCT AgT CAT CCC ACG ATC AGG
AAG ATG A
Primers to make fusion protein construct
SPBN-Nfu + PRPa(core):
PRPc(+) (42-mer) (with BsiWI site)

(SEQ ID NO: 13)

GAG TgT ATA CgT ACg CAA GGA GGG GGT ACC CAT AAT
CAG TGA
PRPc(−) (44-mer) (with NheI site and stop codon)

(SEQ ID NO: 14)

CAT AAg CTA gCT AgC TCA GGA TCT TCT CCC GTC GTA
ATA GGC CT

### EXAMPLE 10

This example illustrates the cell surface expression of the recombinant Anthrax PA protein and the seroconversion of mice immunized with recombinant virions. In order to compare existing and RV-virion based Anthrax vaccines, three groups of 5 Swiss Webster mice were immunized (intramuscularly, i.m.) with recombinant virions containing Anthrax PA63 (D4-E51 killed, 5 micrograms each mouse), wild-type virions (5 micrograms SPBN killed each mouse), or the live RV vector expressing RVG-D4-E51 (3×10⁶ ffu) at week zero (prime) and mice were boosted (boost) three weeks after the initial prime immunization with the same vaccine.

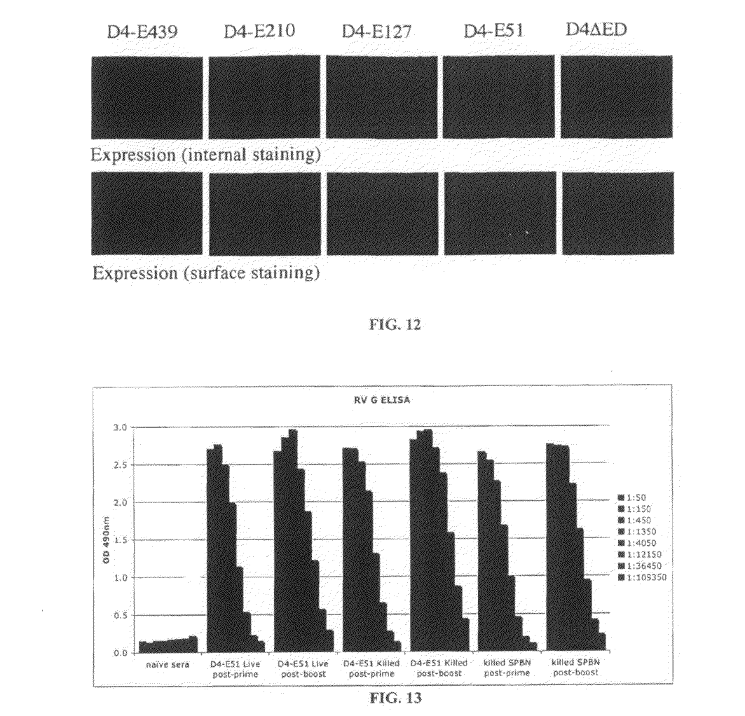

Small samples of blood were extracted retro-orbitally at 5 days prior to immunization, two weeks after the prime, and two weeks after the boost (week 5) to follow the induced Anthrax PA and RV primary-, and secondary immune responses after immunization. (FIG. 13, RV G and FIG. 14 Anthrax PA). These data indicate that one single inoculation with live or killed RV virions containing the chimeric RV G/PA 63-D4 protein induce Anthrax PA-specific seroconversion. A second inoculation increased these responses to high titers.

An ELISA assay for anti-PA or anti RV G antibodies was performed to analyze the immune responses of the immunizations (supra). The RV G protein is isolated from purified virions by well-established methods in the laboratory. Recombinant Anthrax PA is purchased from List Biological Laboratories Inc., Campbell, Calif., U.S.A. Anthrax PA or RV G is resuspended in ELISA plate coating buffer (50 mM Na₂CO₃, pH 9.6) at a concentration of 200 ng/ml and plated in 96-well ELISA MaxiSorp plates (Nunc Inc.) with 100 μl in each well. After overnight incubation at 4° C., plates are washed three times (PBS pH 7.4, 0.1% Tween-20), blocked with blocking buffer (PBS, pH 7.4, 5% dry milk powder) for 30 minutes at room temperature, and incubated with serial dilutions of sera for 1 hour. Plates are washed three times followed by the addition of horseradish peroxidase-conjugated (HRP) goat anti-mouse-IgG (H+ L) secondary antibody (1:5000, Jackson ImmunoResearch Laboratories). After a 30-minute incubation at 37° C., plates are washed three times and 200 μl OPD-substrate (o-phenylenediamine dihydrochloride, Sigma) is added to each well. The reaction is stopped by the addition of 50 μl of 3 M H₂SO₄ per well. Optical density is determined at 490 nm. Monoclonal antibodies directed against Anthrax PA or RV G protein serve as controls.

The results of this example shows that the PA63 D4 construct containing Domain 4 of the Anthrax PA toxin and 51 amino acids of the glycoprotein ectodomain are sufficient for cell surface expression of the foreign antigen (Anthrax PA D4). Shorter or longer portions of the glycoprotein ectodomain may also be used. Immunization of mice with a recombinant virion vaccines containing the D4-E51 construct (Anthrax Domain 4 fused to 51 amino acids of the G ED) resulted in Anthrax specific seroconversion, high titers of protective antibody were detected in the mice (FIGS. 13 and 14). A shorter portion of the ED may also be used, so long as the portion of the ED in the glycoprotein-antigen fusion results in cell surface expression of the foreign antigen.

### EXAMPLE 11

This working example describes the expression of the Botulinum neurotoxin by RV vectors in cells, incorporation of the Botulinum neurotoxin into RNP's and immunogenicity of the recombinant Botulinum neurotoxin A HC50. Recombinant RV's expressing a chimeric RV N-HC50 as an extra gene were constructed as illustrated in FIG. 1E and FIG. 1F. These two different RV's express botulinum neurotoxin domain HC50 as an additional gene (SPBN-N-HC50 in FIG. 1E) and (BNSP-N-HC50 in FIG. 1F). BNSP-N-HC50 is a recombinant RV where the RV N (SEQ ID NO:26) protein is replaced by the RV-N-HC50 protein. In this example, SPBN-N-HC50 was used to analyze if this protein can efficiently be expressed in eukaryotic cells by RV vectors. All methods to have been described previously (supra).

As shown in FIG. 2C by immunostaining with a rabbit antibody directed against Botulinum neurotoxin A, this protein is well expressed with a mainly cytoplasmic location (FIG. 2C). The co-localization with RV N indicates that this protein may be incorporated in RV RNP.

RV N-HC50 fusion protein is incorporated into RV RNPs. As mentioned above, the recombinant RV SPBN-N-HC50 construct was made and recovered from the recombinant Rabies virus, which expressed HC50 fused to RV N as an extra gene. As shown for the RV N-GFP (SEQ ID NO: 30) fusion protein (supra), N-HC50 is incorporated into RV RNPs (FIG. 3D).

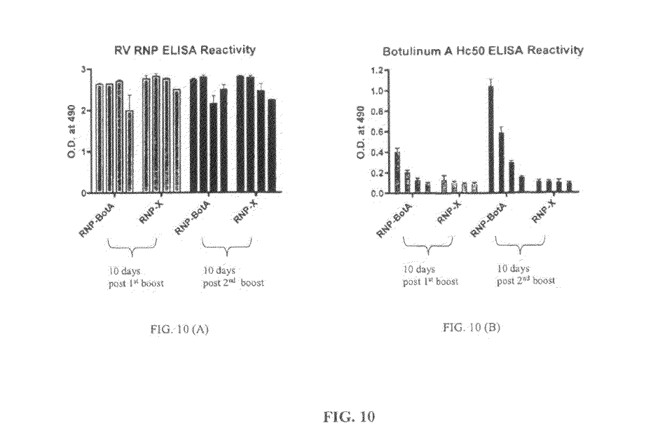

To study the immunogenicity of the recombinant RNPs, as described supra, containing BoNT A HC50, two groups of five six-week-old female Swiss Webster mice were immunized i.m. with 5 μg of recombinant RNPs derived from SPBN-N-HC50 (RNP-BotA), or 5 μg RNPs containing not HC50 as a control (RNP-X). Neither a humoral immune response against RV N or seroconversion against HC50 was detected in any of the two groups of mice after priming. However, the first boost with the same antigens as used for priming resulted in a strong RV N specific immune response in both groups of mice immunized with RNP (FIG. 10(A), RV RNP ELISA Reactivity, red bars (10 days post 1^{st} boost)). In addition, an HC50-specific humoral response was detected in the sera of mice immunized with N-HC50 (FIG. 10(B), RV Botulinum ELISA Reactivity, red bars (10 days post 1^{st} boost)). The second boost (FIG. 10(A), blue bars (10 days post 2^{nd} boost)) further enhanced the RNP specific responses but also resulted in a dramatic increase in HC50 specific responses in the case of RNP-N-HC50 immunized mice. Again, no HC50-specific immune response was detected in mice immunized with the control RNP (FIG. 10(B), RV Botulinum A Hc50 ELISA Reactivity, RNP-X, blue bars (10 days post 2^{nd} boost)).

Although the disclosure has provided considerable detail with reference to certain preferred embodiments thereof, other versions are possible. Therefore the spirit and scope of the appended claims should not be limited to the description and the preferred versions contain within this specification.

TABLE 2

PA63-P
5′-AAACCTAGGATGGGATCCACAAGTGCTGGAC-3′
AVrII site in bold
PA63-M
5′-TTTGTTAACTCCTATCTCATAGCCCTTCTTAG-3′
HpaI site in bold
D4-P
5′-AAACCTAGGGACAAGCGTTTCCATTATGATA-3′
SpeI in bold
D4-M
5′-TTTGTTAACTCTAGATCCTATCTCATAGCCCTTCTTAG-3′
HpaI and XbaI, bold
RV-ED439
5′-CCCTCTAGAAAATTCCCTATTTACACGATACC-3′
XbaI, bold
RV-ED
210′-CCCTCTAGAGTTCTAGGACTTAGACTTATGG-3′
XbaI, bold
RV-ED
127′-CCCTCTAGAAAAGCATATACCATATTCAACAAG-3′
XbaI, bold
RV-ED51
5′-CCCTCTAGAGAATCCTCGGTTATCCCCCTTG-3′
XbaI, bold
RVG-ED-M
5′-TTTGTTAACCTTCCCCCAGTTCGGGAGACC-3′
HpaI, bold

What is claimed is:

1. A composition comprising:
a recombinant Nfu-antigen fusion protein expressed by negative-stranded RNA virus.

2. The composition of claim 1 wherein the negative-stranded RNA virus is a rhabdovirus.

3. The composition of claim 1 wherein the negative stranded RNA virus is a Lyssavirus.

4. The composition of claim 1 wherein the protein antigen fusion expressed by said negative stranded RNA virus is an SPBN-Nfu-antigen virus.

5. A composition comprising:
an expressed Rhabdovirus nucleoprotein-antigen fusion protein wherein a nucleoprotein portion has an authentic amino terminus, and an antigen portion is fused to a carboxy terminus of said nucleoprotein portion of the nucleoprotein-antigen fusion protein of said Rhabdovirus.

6. The composition of claim 5 wherein the nucleoprotein-antigen fusion protein is expressed by a recombinant Rhabdovirus.

7. The composition of claim 5 wherein the nucleoprotein-antigen fusion protein is expressed by a SPBN-Nfu-antigen virus

8. A method of screening recombinant negative strand viral RNPs comprising:
immunizing one or more groups of test subjects, each group immunized with a recombinant Nfu-antigen fusion protein expressed by a negative-strand RNA virus;
measuring humoral responses on test subjects in each group; and
selecting a recombinant Nfu-antigen fusion protein expressed by the negative-strand RNA virus that provides greatest humoral response for use in a pharmaceutical composition or vaccine against an antigen portion in the Nfu-antigen fusion protein.

9. The method of claim 8 wherein a response of subjects further includes performing at least one of macrophage cytotoxicity assays, mouse lethal toxin challenge, or a combination of these.

10. A method of making a non-segmented negative-stranded RNA virus entirely from cDNA comprising:
intracellularly expressing antigenomic RNA in cells also expressing viral proteins for formation of an active RNP complex.

11. The method of claim 10 wherein the cell expressed viral proteins comprise: a nucleoprotein (N), a phosphoprotein (P), and a viral polymerase (L).

12. A composition comprising:
a plasmid encoding an RV G signal sequence, an RV G ectodomain sequence, an antigen sequence, an RV G transmembrane domain, and a cytoplasmic domain.

13. The composition of claim 12 wherein said RV G ectodomain sequence encodes for about 51 amino acids.

14. The composition of claim 12 wherein the foreign antigen sequence is from Anthrax or Botulin.

15. The composition of claim 12 wherein the plasmid is transfected into BSR cells for recovery of a recombinant virus.

16. A composition comprising:
a plasmid encoding an RV Nfu sequence or its variants and an antigen sequence.

17. The composition of claim 16 wherein the RV Nfu sequence has an N protein amino terminus.

18. The composition of claim 16 wherein the RV Nfu sequence has the antigen sequence at the carboxy terminus of the N protein.

19. The composition of claim 16 wherein the plasmid is transfected into BSR cells for recovery of a recombinant virus.

20. A composition comprising:
a chimeric glycoprotein-antigen fusion protein; an amino terminal portion of the antigen in the fusion protein having a viral glycoprotein signal sequence fused to the antigen, and a carboxy terminal portion of the antigen in the fusion protein having an ectodomain portion, a transmembrane domain portion, and a cytoplasmic domain portion of the viral glycoprotein.

21. The composition of claim 20 wherein the chimeric glycoprotein antigen fusion protein is incorporated into a viral membrane.

22. The composition of claim 20 wherein the viral glycoprotein is from a Rhabdovirus.

23. The composition of claim 20 wherein the viral glycoprotein is from a Lyssavirus.

24. The composition of claim 20 wherein the viral glycoprotein is from a Rabies virus.

25. The composition of claim 20 wherein the antigen portion in the fusion protein is a variant or subdomain of a full length antigen.

26. The composition of claim 25 wherein the subdomain of a full length antigen is domain 4 of an anthrax PA protein.

27. The composition of claim 20 wherein the ectodomain portion is that of a rabies virus glycoprotein.

28. The composition of claim 20 wherein the ectodomain portion has one or more amino acids removed and can transport the chimeric glycoprotein-antigen to the cell surface.

29. The composition of claim 28 wherein the ectodomain has about 51 amino acids.

30. An expression plasmid encoding a chimeric glycoprotein-antigen fusion protein having an amino terminal portion of the antigen in the fusion protein having a viral glycoprotein signal sequence fused to the antigen, and a carboxy terminal portion of the antigen in the fusion protein having an ectodomain portion, a transmembrane domain portion, and a cytoplasmic domain portion of the viral glycoprotein.

31. The expression plasmid of claim 30 wherein the ectodomain portion is that of a rabies virus glycoprotein.

32. The expression plasmid of claim 31 wherein the ectodomain portion has or more amino acids removed and can transport the chimeric glycoprotein-antigen to the cell surface.

33. The expression plasmid of claim 30 wherein the antigen in the fusion protein is domain 4 of an anthrax PA protein.

34. A method for immunizing comprising: administering to an individual an amount of at least one fusion protein expressed by a modified N gene or a modified G gene with a modified ectodomain, of a rabies virus, the fusion protein comprises at least a portion of the N protein or G protein with the modified ectodomain of the rabies virus linked to an immunogenic portion of an antigen, the amount of the fusion protein being effective to elicit a B cell-mediated humoral immune response in a subject.

35. The method of claim 34 where the antigen is a bacterial toxin or variant of the bacterial toxin.

36. The method of claim 35 where the bacterial toxin is a portion of an anthrax, botulin, or cholera toxin.

37. The method of claim 35 where the bacterial toxin is a portion of anthrax protective antigen.

38. The method of claim 35 where the bacterial toxin is a portion of botulin toxin.

39. A composition comprising: a rabies virus comprising a gene encoding an N protein or a gene encoding a G protein with a modified ectodomain fused to an antigen, or variant of the antigen, the rabies virus N protein has an authentic amino terminus.

40. The composition of claim 39 where the antigen is a bacterial toxin.

41. The composition of claim 39 where the antigen is HIV-1 Env epitopes and protein, Hepatitis B surface antigen; Abrin, Aflatoxins, Botulinum toxin, Anthrax protective antigen, Cholera toxin, Clostridium perfringens, Conotoxin, Diacetoxyscirpenol toxin, HT-2 toxin, Microcystin, Modeccin toxin, Ricin, Saxitoxin, Shiga toxin, Staphylococcus aureus toxin, or variant of these.

42. The composition of claim 39 where the antigen is: CA-125: a protein on the surface of ovarian cancer cells; CA 15-3: a high molecular weight protein associated with breast cancer cells; PSA (prostate specific antigen): a protein associated with prostate cancer cells; and Mesothelin: a protein over expressed in primary pancreatic and ovarian cancer cells, and variants of these antigens.

43. A composition comprising: a rabies virus protein including at least a portion of an N protein or a G protein fused to a bacterial antigen, viral antigen, or cancer antigen, or variant of the antigen.

44. The composition of claim 43 where the antigen is a bacterial toxin.

45. The composition of claim 43 where the antigen is HIV-1 Env epitopes and protein, Hepatitis B surface antigen; Abrin, Aflatoxins, Botulinum toxin, anthrax protective antigen, Cholera toxin, Clostridium perfringens, Conotoxin, Diacetoxyscirpenol toxin, HT-2 toxin, Microcystin, Modeccin toxin, Ricin, Saxitoxin, Shiga toxin, Staphylococcus aureus toxin, or variants of these.

46. The composition of claim 43 where the cancer antigen is: CA-125: a protein on the surface of ovarian cancer cells; CA 15-3: a high molecular weight protein associated with breast cancer cells; PSA (prostate specific antigen): a protein associated with prostate cancer cells; and Mesothelin: a protein over expressed in primary pancreatic and ovarian cancer cells, and variants of these antigens.

47. A composition comprising: a gene from a rabies virus encoding a bacterial antigen, viral antigen, or cancer antigen fused to at least a portion of an N protein or a G protein; the rabies virus has an authentic amino terminus.

48. The composition of claim 47 where the antigen is a bacterial toxin.

47. The composition of claim 47 where the antigen is an HIV-1 Env epitope, Hepatitis B surface antigen; Abrin, Aflatoxins, Botulinum toxin, anthrax protective antigen, Cholera toxin, Clostridium perfringens, Conotoxin, Diacetoxyscirpenol toxin, HT-2 toxin, Microcystin, Modeccin toxin, Ricin, Saxitoxin, Shiga toxin, or Staphylococcus aureus toxin, or variants of these.

48. The composition of claim 47 where the cancer antigen is CA-125: a protein on the surface of ovarian cancer cells; CA 15-3: a high molecular weight protein associated with breast cancer cells; PSA (prostate specific antigen): a protein associated with prostate cancer cells; and Mesothelin: a protein over expressed in primary pancreatic and ovarian cancer cells, and variants of these antigens.


SEQUENCE LISTING (34 sequences)

SEQ ID NO: 1 - DNA, 32 bp, Artificial
  Primer for constructing recombinant rabies viruses
aaaacgtacgtgagtcactcgaatatgtcttg

SEQ ID NO: 2 - DNA, 20 bp, Artificial
  Primer for constructing recombinant rabies viruses
tgtgctgcaaggcgattaag

SEQ ID NO: 3 - DNA, 30 bp, Artificial
  Primer for constructing recombinant rabies viruses
tttcgtacggtgagcaagggcgaggagctg

SEQ ID NO: 4 - DNA, 31 bp, Artificial
  Primer for constructing recombinant rabies viruses
aaagctagcttacttgtacagctcgtccatg

SEQ ID NO: 5 - DNA, 31 bp, Artificial
  Primer for constructing recombinant rabies viruses
tttggtaccacaatggatgccgacaagattg

SEQ ID NO: 6 - DNA, 31 bp, Artificial
  Primer for sequencing recombinant rabies v iruses
aaagctagcttatcctatctcatagcccttc

SEQ ID NO: 7 - DNA, 35 bp, Artificial
  Primer for sequencing recombinant rabies v iruses
tttcgtacgacaatgtccacaagtgctggacctac

SEQ ID NO: 8 - DNA, 31 bp, Artificial
  Primer for sequencing recombinant rabies v iruses
tttcgtacggacaagcgtttccattatgata

SEQ ID NO: 9 - DNA, 765 bp, Mouse Prion
atggcgaaccttggctactggctgctggccctctttgtgactatgtggactgatgtcggcctctgcaaaaagcggccaaagcctggagggtggaacaccggtggaagccggtatcccgggcagggaagccctggaggcaaccgttacccacctcagggtggcacctgggggcagccccacggtggtggctggggacaaccccatgggggcagctggggacaacctcatggtggtagttggggtcagccccatggcggtggatggggccaaggagggggtacccataatcagtggaacaagcccagcaaaccaaaaaccaacctcaagcatgtggcaggggctgcggcagctggggcagtagtggggggccttggtggctacatgctggggagcgccatgagcaggcccatgatccattttggcaacgactgggaggaccgctactaccgtgaaaacatgtaccgctaccctaaccaagtgtactacaggccagtggatcagtacagcaaccagaacaacttcgtgcacgactgcgtcaatatcaccatcaagcagcacacggtcaccaccaccaccaagggggagaacttcaccgagaccgatgtgaagatgatggagcgcgtggtggagcagatgtgcgtcacccagtaccagaaggagtcccaggcctattacgacgggagaagatccagcagcaccgtgcttttctcctcccctcctgtcatcctcctcatctccttcctcatcttcctgatcgtgggatga

SEQ ID NO: 10 - DNA, 423 bp, Mouse Prion
caaggagggggtacccataatcagtggaacaagcccagcaaaccaaaaaccaacctcaagcatgtggcaggggctgcggcagctggggcagtagtggggggccttggtggctacatgctggggagcgccatgagcaggcccatgatccattttggcaacgactgggaggaccgctactaccgtgaaaacatgtaccgctaccctaaccaagtgtactacaggccagtggatcagtacagcaaccagaacaacttcgtgcacgactgcgtcaatatcaccatcaagcagcacacggtcaccaccaccaccaagggggagaacttcaccgagaccgatgtgaagatgatggagcgcgtggtggagcagatgtgcgtcacccagtaccagaaggagtcccaggcctattacgacgggagaagatcc

SEQ ID NO: 11 - DNA, 42 bp, Artificial
  Primers used to construct recombinant viruses with the prion protein or core of the prion protein
agatgtatacgtacgaagatggcgaaccttggctactggctg

SEQ ID NO: 12 - DNA, 43 bp, Artificial
  Primers used to construct recombinant viruses with the core of the prion protein
tctatcagctagctagctagtcatcccacgatcaggaagatga

SEQ ID NO: 13 - DNA, 42 bp, Artificial
  Primers used to construct recombinant viruses with the prion protein or core of the prion protein
gagtgtatacgtacgcaaggagggggtacccataatcagtga

SEQ ID NO: 14 - DNA, 44 bp, Artificial
  Primers used to construct recombinant viruses with the prion protein or core of the prion protein
cataagctagctagctcaggatcttctcccgtcgtaataggcct

SEQ ID NO: 15 - DNA, 38 bp, Artificial
  Other primers used to construct the c himeric D4 recombinant viruses with different portions of the ED
tttgttaactctagatcctatctcatagcccttcttag

SEQ ID NO: 16 - DNA, 32 bp, Artificial
  Other primers used to construct the c himeric D4 recombinant viruses with different portions of the ED
ccctctagaaaattccctatttacacgatacc

SEQ ID NO: 17 - DNA, 31 bp, Artificial
  Other primers used to construct the c himeric D4 recombinant viruses with different portions of the ED
ccctctagagttctaggacttagacttatgg

SEQ ID NO: 18 - DNA, 33 bp, Artificial
  Other primers used to construct the c himeric D4 recombinant viruses with different portions of the ED
ccctctagaaaagcatataccatattcaacaag

SEQ ID NO: 19 - DNA, 31 bp, Artificial
  Other primers used to construct the c himeric D4 recombinant viruses with different portions of the ED
ccctctagagaatcctcggttatcccccttg

SEQ ID NO: 20 - DNA, 30 bp, Artificial
  Other primers used to construct the c himeric D4 recombinant viruses with different portions of the ED
tttgttaaccttcccccagttcgggagacc

SEQ ID NO: 21 - DNA, 31 bp, Artificial; identical to SEQ ID NO: 6
  Nucleic acid primer PA63-Nfu-M used to construct a modified rabies virus

SEQ ID NO: 22 - DNA, 35 bp, Artificial; identical to SEQ ID NO: 7
  Nucleic acid primer PA63-Nfu-P used to construct a modified rabies virus

SEQ ID NO: 23 - DNA, 31 bp, Artificial; identical to SEQ ID NO: 8
  Nucleic acid primer D4-Nfu-P used to construct a modified rabies virus

SEQ ID NO: 24 - DNA, 11574 bp, Artificial
  Rabies virus vector
acgcttaacaaccagatcaaagaaaaaacagacattgtcaattgcaaagcaaaaatgtaacacccctacaatggatgccgacaagattgtattcaaagtcaataatcaggtggtctctttgaagcctgagattatcgtggatcaatatgagtacaagtaccctgccatcaaagatttgaaaaagccctgtataaccctaggaaaggctcccgatttaaataaagcatacaagtcagttttgtcaggcatgagcgccgccaaacttaatcctgacgatgtatgttcctatttggcagcggcaatgcagttttttgaggggacatgtccggaagactggaccagctatggaattgtgattgcacgaaaaggagataagatcaccccaggttctctggtggagataaaacgtactgatgtagaagggaattgggctctgacaggaggcatggaactgacaagagaccccactgtccctgagcatgcgtccttagtcggtcttctcttgagtctgtataggttgagcaaaatatccgggcaaaacactggtaactataagacaaacattgcagacaggatagagcagatttttgagacagccccttttgttaaaatcgtggaacaccatactctaatgacaactcacaaaatgtgtgctaattggagtactataccaaacttcagatttttggccggaacctatgacatgtttttctcccggattgagcatctatattcagcaatcagagtgggcacagttgtcactgcttatgaagactgttcaggactggtatcatttactgggttcataaaacaaatcaatctcaccgctagagaggcaatactatatttcttccacaagaactttgaggaagagataagaagaatgtttgagccagggcaggagacagctgttcctcactcttatttcatccacttccgttcactaggcttgagtgggaaatctccttattcatcaaatgctgttggtcacgtgttcaatctcattcactttgtaggatgctatatgggtcaagtcagatccctaaatgcaacggttattgctgcatgtgctcctcatgaaatgtctgttctagggggctatctgggagaggaattcttcgggaaagggacatttgaaagaagattcttcagagatgagaaagaacttcaagaatacgaggcggctgaactgacaaagactgacgtagcactggcagatgatggaactgtcaactctgacgacgaggactacttttcaggtgaaaccagaagtccggaggctgtttatactcgaatcatgatgaatggaggtcgactaaagagatctcacatacggagatatgtctcagtcagttccaatcatcaagcccgtccaaactcattcgccgagtttctaaacaagacatattcgagtgactcataagaagttgaataacaaaatgccggaaatctacggattgtgtatatccatcatgaaaaaaactaacacccctcctttcgaaccatcccaaacatgagcaagatctttgtcaatcctagtgctattagagccggtctggccgatcttgagatggctgaagaaactgttgatctgatcaatagaaatatcgaagacaatcaggctcatctccaaggggaacccatagaggtggacaatctccctgaggatatggggcgacttcacctggatgatggaaaatcgcccaaccatggtgagatagccaaggtgggagaaggcaagtatcgagaggactttcagatggatgaaggagaggatcctagcttcctgttccagtcatacctggaaaatgttggagtccaaatagtcagacaaatgaggtcaggagagagatttctcaagatatggtcacagaccgtagaagagattatatcctatgtcgcggtcaactttcccaaccctccaggaaagtcttcagaggataaatcaacccagactactggccgagagctcaagaaggagacaacacccactccttctcagagagaaagccaatcatcgaaagccaggatggcggctcaaattgcttctggccctccagcccttgaatggtcggctaccaatgaagaggatgatctatcagtggaggctgagatcgctcaccagattgcagaaagtttctccaaaaaatataagtttccctctcgatcctcagggatactcttgtataattttgagcaattgaaaatgaaccttgatgatatagttaaagaggcaaaaaatgtaccaggtgtgacccgtttagcccatgacgggtccaaactccccctaagatgtgtactgggatgggtcgctttggccaactctaagaaattccagttgttagtcgaatccgacaagctgagtaaaatcatgcaagatgacttgaatcgctatacatcttgctaaccgaacctctcccctcagtccctctagacaataaaatccgagatgtcccaaagtcaacatgaaaaaaacaggcaacaccactgataaaatgaacctcctacgtaagatagtgaaaaaccgcagggacgaggacactcaaaaatcctctcccgcgtcagcccctctggatgacgatgacttgtggcttccaccccctgaatacgtcccgctgaaagaacttacaggcaagaagaacatgaggaacttttgtatcaacggaagggttaaagtgtgtagcccgaatggttactcgttcaggatcctgcggcacattctgaaatcattcgacgagatatattctgggaatcataggatgatcgggttagtcaaagtggttattggactggctttgtcaggatctccagtccctgagggcctgaactgggtatacaaattgaggagaacctttatcttccagtgggctgattccaggggccctcttgaaggggaggagttggaatactctcaggagatcacttgggatgatgatactgagttcgtcggattgcaaataagagtgattgcaaaacagtgtcatatccagggcagagtctggtgtatcaacatgaacccgagagcatgtcaactatggtctgacatgtctcttcagacacaaaggtccgaagaggacaaagattcctctctgcttctagaataatcagattatatcccgcaaatttatcacttgtttacctctggaggagagaacatatgggctcaactccaacccttgggagcaatataacaaaaaacatgttatggtgccattaaaccgctgcatttcatcaaagtcaagttgattacctttacattttgatcctcttggatgtgaaaaaaactattaacatccctcaaaagaccccgggaaagatggttcctcaggctctcctgtttgtaccccttctggtttttccattgtgttttgggaaattccctatttacacgataccagacaagcttggtccctggagtccgattgacatacatcacctcagctgcccaaacaatttggtagtggaggacgaaggatgcaccaacctgtcagggttctcctacatggaacttaaagttggatacatcttagccataaaagtgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtggcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtctcgatgcaaacatcaaatgaaaccaaatggtgccctcccgataagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcagaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtaattaattaacgtcctttcaacgatccaagtccatgaaaaaaactaacacccctcccgtacgttttgctagcttataaagtgctgggtcatctaagcttttcagtcgagaaaaaaacattagatcagaagaacaactggcaacacttctcaacctgagacttacttcaagatgctcgatcctggagaggtctatgatgaccctattgacccaatcgagttagaggctgaacccagaggaacccccattgtccccaacatcttgaggaactctgactacaatctcaactctcctttgatagaagatcctgctagactaatgttagaatggttaaaaacagggaatagaccttatcggatgactctaacagacaattgctccaggtctttcagagttttgaaagattatttcaagaaggtagatttgggttctctcaaggtgggcggaatggctgcacagtcaatgatttctctctggttatatggtgcccactctgaatccaacaggagccggagatgtataacagacttggcccatttctattccaagtcgtcccccatagagaagctgttgaatctcacgctaggaaatagagggctgagaatccccccagagggagtgttaagttgccttgagagggttgattatgataatgcatttggaaggtatcttgccaacacgtattcctcttacttgttcttccatgtaatcaccttatacatgaacgccctagactgggatgaagaaaagaccatcctagcattatggaaagatttaacctcagtggacatcgggaaggacttggtaaagttcaaagaccaaatatggggactgctgatcgtgacaaaggactttgtttactcccaaagttccaattgtctttttgacagaaactacacacttatgctaaaagatcttttcttgtctcgcttcaactccttaatggtcttgctctctcccccagagccccgatactcagatgacttgatatctcaactatgccagctgtacattgctggggatcaagtcttgtctatgtgtggaaactccggctatgaagtcatcaaaatattggagccatatgtcgtgaatagtttagtccagagagcagaaaagtttaggcctctcattcattccttgggagactttcctgtatttataaaagacaaggtaagtcaacttgaagagacgttcggtccctgtgcaagaaggttctttagggctctggatcaattcgacaacatacatgacttggtttttgtgtttggctgttacaggcattgggggcacccatatatagattatcgaaagggtctgtcaaaactatatgatcaggttcaccttaaaaaaatgatagataagtcctaccaggagtgcttagcaagcgacctagccaggaggatccttagatggggttttgataagtactccaagtggtatctggattcaagattcctagcccgagaccaccccttgactccttatatcaaaacccaaacatggccacccaaacatattgtagacttggtgggggatacatggcacaagctcccgatcacgcagatctttgagattcctgaatcaatggatccgtcagaaatattggatgacaaatcacattctttcaccagaacgagactagcttcttggctgtcagaaaaccgaggggggcctgttcctagcgaaaaagttattatcacggccctgtctaagccgcctgtcaatccccgagagtttctgaggtctatagacctcggaggattgccagatgaagacttgataattggcctcaagccaaaggaacgggaattgaagattgaaggtcgattctttgctctaatgtcatggaatctaagattgtattttgtcatcactgaaaaactcttggccaactacatcttgccactttttgacgcgctgactatgacagacaacctgaacaaggtgtttaaaaagctgatcgacagggtcaccgggcaagggcttttggactattcaagggtcacatatgcatttcacctggactatgaaaagtggaacaaccatcaaagattagagtcaacagaggatgtattttctgtcctagatcaagtgtttggattgaagagagtgttttctagaacacacgagttttttcaaaaggcctggatctattattcagacagatcagacctcatcgggttacgggaggatcaaatatactgcttagatgcgtccaacggcccaacctgttggaatggccaggatggcgggctagaaggcttacggcagaagggctggagtctagtcagcttattgatgatagatagagaatctcaaatcaggaacacaagaaccaaaatactagctcaaggagacaaccaggttttatgtccgacatacatgttgtcgccagggctatctcaagaggggctcctctatgaattggagagaatatcaaggaatgcactttcgatatacagagccgtcgaggaaggggcatctaagctagggctgatcatcaagaaagaagagaccatgtgtagttatgacttcctcatctatggaaaaacccctttgtttagaggtaacatattggtgcctgagtccaaaagatgggccagagtctcttgcgtctctaatgaccaaatagtcaacctcgccaatataatgtcgacagtgtccaccaatgcgctaacagtggcacaacactctcaatctttgatcaaaccgatgagggattttctgctcatgtcagtacaggcagtctttcactacctgctatttagcccaatcttaaagggaagagtttacaagattctgagcgctgaaggggagagctttctcctagccatgtcaaggataatctatctagatccttctttgggagggatatctggaatgtccctcggaagattccatatacgacagttctcagaccctgtctctgaagggttatccttctggagagagatctggttaagctcccaagagtcctggattcacgcgttgtgtcaagaggctggaaacccagatcttggagagagaacactcgagagcttcactcgccttctagaagatccgaccaccttaaatatcagaggaggggccagtcctaccattctactcaaggatgcaatcagaaaggctttatatgacgaggtggacaaggtggaaaattcagagtttcgagaggcaatcctgttgtccaagacccatagagataattttatactcttcttaatatctgttgagcctctgtttcctcgatttctcagtgagctattcagttcgtcttttttgggaatccccgagtcaatcattggattgatacaaaactcccgaacgataagaaggcagtttagaaagagtctctcaaaaactttagaagaatccttctacaactcagagatccacgggattagtcggatgacccagacacctcagagggttgggggggtgtggccttgctcttcagagagggcagatctacttagggagatctcttggggaagaaaagtggtaggcacgacagttcctcacccttctgagatgttgggattacttcccaagtcctctatttcttgcacttgtggagcaacaggaggaggcaatcctagagtttctgtatcagtactcccgtcctttgatcagtcatttttttcacgaggccccctaaagggatacttgggctcgtccacctctatgtcgacccagctattccatgcatgggaaaaagtcactaatgttcatgtggtgaagagagctctatcgttaaaagaatctataaactggttcattactagagattccaacttggctcaagctctaattaggaacattatgtctctgacaggccctgatttccctctagaggaggcccctgtcttcaaaaggacggggtcagccttgcataggttcaagtctgccagatacagcgaaggagggtattcttctgtctgcccgaacctcctctctcatatttctgttagtacagacaccatgtctgatttgacccaagacgggaagaactacgatttcatgttccagccattgatgctttatgcacagacatggacatcagagctggtacagagagacacaaggctaagagactctacgtttcattggcacctccgatgcaacaggtgtgtgagacccattgacgacgtgaccctggagacctctcagatcttcgagtttccggatgtgtcgaaaagaatatccagaatggtttctggggctgtgcctcacttccagaggcttcccgatatccgtctgagaccaggagattttgaatctctaagcggtagagaaaagtctcaccatatcggatcagctcaggggctcttatactcaatcttagtggcaattcacgactcaggatacaatgatggaaccatcttccctgtcaacatatacggcaaggtttcccctagagactatttgagagggctcgcaaggggagtattgataggatcctcgatttgcttcttgacaagaatgacaaatatcaatattaatagacctcttgaattggtctcaggggtaatctcatatattctcctgaggctagataaccatccctccttgtacataatgctcagagaaccgtctcttagaggagagatattttctatccctcagaaaatccccgccgcttatccaaccactatgaaagaaggcaacagatcaatcttgtgttatctccaacatgtgctacgctatgagcgagagataatcacggcgtctccagagaatgactggctatggatcttttcagactttagaagtgccaaaatgacgtacctatccctcattacttaccagtctcatcttctactccagagggttgagagaaacctatctaagagtatgagagataacctgcgacaattgagttctttgatgaggcaggtgctgggcgggcacggagaagataccttagagtcagacgacaacattcaacgactgctaaaagactctttacgaaggacaagatgggtggatcaagaggtgcgccatgcagctagaaccatgactggagattacagccccaacaagaaggtgtcccgtaaggtaggatgttcagaatgggtctgctctgctcaacaggttgcagtctctacctcagcaaacccggcccctgtctcggagcttgacataagggccctctctaagaggttccagaaccctttgatctcgggcttgagagtggttcagtgggcaaccggtgctcattataagcttaagcctattctagatgatctcaatgttttcccatctctctgccttgtagttggggacgggtcaggggggatatcaagggcagtcctcaacatgtttccagatgccaagcttgtgttcaacagtcttttagaggtgaatgacctgatggcttccggaacacatccactgcctccttcagcaatcatgaggggaggaaatgatatcgtctccagagtgatagatcttgactcaatctgggaaaaaccgtccgacttgagaaacttggcaacctggaaatacttccagtcagtccaaaagcaggtcaacatgtcctatgacctcattatttgcgatgcagaagttactgacattgcatctatcaaccggatcaccctgttaatgtccgattttgcattgtctatagatggaccactctatttggtcttcaaaacttatgggactatgctagtaaatccaaactacaaggctattcaacacctgtcaagagcgttcccctcggtcacagggtttatcacccaagtaacttcgtctttttcatctgagctctacctccgattctccaaacgagggaagtttttcagagatgctgagtacttgacctcttccacccttcgagaaatgagccttgtgttattcaattgtagcagccccaagagtgagatgcagagagctcgttccttgaactatcaggatcttgtgagaggatttcctgaagaaatcatatcaaatccttacaatgagatgatcataactctgattgacagtgatgtagaatcttttctagtccacaagatggttgatgatcttgagttacagaggggaactctgtctaaagtggctatcattatagccatcatgatagttttctccaacagagtcttcaacgtttccaaacccctaactgacccctcgttctatccaccgtctgatcccaaaatcctgaggcacttcaacatatgttgcagtactatgatgtatctatctactgctttaggtgacgtccctagcttcgcaagacttcacgacctgtataacagacctataacttattacttcagaaagcaagtcattcgagggaacgtttatctatcttggagttggtccaacgacacctcagtgttcaaaagggtagcctgtaattctagcctgagtctgtcatctcactggatcaggttgatttacaagatagtgaagactaccagactcgttggcagcatcaaggatctatccagagaagtggaaagacaccttcataggtacaacaggtggatcaccctagaggatatcagatctagatcatccctactagactacagttgcctgtgaaccggatactcctggaagcctgcccatgctaagactcttgtgtgatgtatcttgaaaaaaacaagatcctaaatctgaacctttggttgtttgattgtttttctcatttttgttgtttatttgttaagcgt

SEQ ID NO: 25 - DNA, 1353 bp, Artificial
  Rabies virus N sequence
atggatgccgacaagattgtattcaaagtcaataatcaggtggtctctttgaagcctgagattatcgtggatcaatatgagtacaagtaccctgccatcaaagatttgaaaaagccctgtataaccctaggaaaggctcccgatttaaataaagcatacaagtcagttttgtcaggcatgagcgccgccaaacttaatcctgacgatgtatgttcctatttggcagcggcaatgcagttttttgaggggacatgtccggaagactggaccagctatggaattgtgattgcacgaaaaggagataagatcaccccaggttctctggtggagataaaacgtactgatgtagaagggaattgggctctgacaggaggcatggaactgacaagagaccccactgtccctgagcatgcgtccttagtcggtcttctcttgagtctgtataggttgagcaaaatatccgggcaaaacactggtaactataagacaaacattgcagacaggatagagcagatttttgagacagccccttttgttaaaatcgtggaacaccatactctaatgacaactcacaaaatgtgtgctaattggagtactataccaaacttcagatttttggccggaacctatgacatgtttttctcccggattgagcatctatattcagcaatcagagtgggcacagttgtcactgcttatgaagactgttcaggactggtatcatttactgggttcataaaacaaatcaatctcaccgctagagaggcaatactatatttcttccacaagaactttgaggaagagataagaagaatgtttgagccagggcaggagacagctgttcctcactcttatttcatccacttccgttcactaggcttgagtgggaaatctccttattcatcaaatgctgttggtcacgtgttcaatctcattcactttgtaggatgctatatgggtcaagtcagatccctaaatgcaacggttattgctgcatgtgctcctcatgaaatgtctgttctagggggctatctgggagaggaattcttcgggaaagggacatttgaaagaagattcttcagagatgagaaagaacttcaagaatacgaggcggctgaactgacaaagactgacgtagcactggcagatgatggaactgtcaactctgacgacgaggactacttttcaggtgaaaccagaagtccggaggctgtttatactcgaatcatgatgaatggaggtcgactaaagagatctcacatacggagatatgtctcagtcagttccaatcatcaagcccgtccaaactcattcgccgagtttctaaacaagacatattcgagtgactcataa

SEQ ID NO: 26 - protein, 450 aa, Artificial
  Protein expressed by modified rabies virus N protein sequence
MDADKIVFKVNNQVVSLKPEIIVDQYEYKYPAIKDLKKPCITLGKAPDLNKAYKSVLSGMSAAKLNPDDVCSYLAAAMQFFEGTCPEDWTSYGIVIARKGDKITPGSLVEIKRTDVEGNWALTGGMELTRDPTVPEHASLVGLLLSLYRLSKISGQNTGNYKTNIADRIEQIFETAPFVKIVEHHTLMTTHKMCANWSTIPNFRFLAGTYDMFFSRIEHLYSAIRVGTVVTAYEDCSGLVSFTGFIKQINLTAREAILYFFHKNFEEEIRRMFEPGQETAVPHSYFIHFRSLGLSGKSPYSSNAVGHVFNLIHFVGCYMGQVRSLNATVIAACAPHEMSVLGGYLGEEFFGKGTFERRFFRDEKELQEYEAAELTKTDVALADDGTVNSDDEDYFSGETRSPEAVYTRIMMNGGRLKRSHIRRYVSVSSNHQARPNSFAEFLNKTYSSDS

SEQ ID NO: 27 - DNA, 3069 bp, Artificial
  Nucleic acid of modified rabies virus expressing N-Fu-PA63
atggatgccgacaagattgtattcaaagtcaataatcaggtggtctctttgaagcctgagattatcgtggatcaatatgagtacaagtaccctgccatcaaagatttgaaaaagccctgtataaccctaggaaaggctcccgatttaaataaagcatacaagtcagttttgtcaggcatgagcgccgccaaacttaatcctgacgatgtatgttcctatttggcagcggcaatgcagttttttgaggggacatgtccggaagactggaccagctatggaattgtgattgcacgaaaaggagataagatcaccccaggttctctggtggagataaaacgtactgatgtagaagggaattgggctctgacaggaggcatggaactgacaagagaccccactgtccctgagcatgcgtccttagtcggtcttctcttgagtctgtataggttgagcaaaatatccgggcaaaacactggtaactataagacaaacattgcagacaggatagagcagatttttgagacagccccttttgttaaaatcgtggaacaccatactctaatgacaactcacaaaatgtgtgctaattggagtactataccaaacttcagatttttggccggaacctatgacatgtttttctcccggattgagcatctatattcagcaatcagagtgggcacagttgtcactgcttatgaagactgttcaggactggtatcatttactgggttcataaaacaaatcaatctcaccgctagagaggcaatactatatttcttccacaagaactttgaggaagagataagaagaatgtttgagccagggcaggagacagctgttcctcactcttatttcatccacttccgttcactaggcttgagtgggaaatctccttattcatcaaatgctgttggtcacgtgttcaatctcattcactttgtaggatgctatatgggtcaagtcagatccctaaatgcaacggttattgctgcatgtgctcctcatgaaatgtctgttctagggggctatctgggagaggaattcttcgggaaagggacatttgaaagaagattcttcagagatgagaaagaacttcaagaatacgaggcggctgaactgacaaagactgacgtagcactggcagatgatggaactgtcaactctgacgacgaggactacttttcaggtgaaaccagaagtccggaggctgtttatactcgaatcatgatgaatggaggtcgactaaagagatctcacatacggagatatgtctcagtcagttccaatcatcaagcccgtccaaactcattcgccgagtttctaaacaagacatattcgagtgactcacgtacgacaatgtccacaagtgctggacctacggttccagaccgtgacaatgatggaatccctgattcattagaggtagagggatatacggttgacgtcaaaaataaaagaacttttctttcaccatggatttctaacattcacgagaagaagggattaaccaagtataaatcatctcctgaaaagtggagcacggcttctgacccgtacagtgacttcgagaaggttacaggacggattgataagaatgtatcaccagaggcaagacacccccttgtggcagcttatccgattgtacacgtagacatggagaatattattctctcaaagaatgaggatcaatccacacagaacactgacagtgaaacgagaacaataagtaagaatacttctacaagtaggacacatactagtgaagtacacggaaacgcagaagtgcatgcgtcgttctttgatattggtgggagtgtatctgcaggattcagtaactcgaattcaagtacggtcgcaattgaccattcactatctctagcaggggagagaacttgggctgaaacaatgggtttaaacaccgctgacacagcaagattaaatgccaatattagatatgtaaatactgggacggctccaatctacaacgtgttaccaacgacttcgttagtgttaggaaagaatcagacactcgcgacaattaaggctgacgagaaccaattaagtcaaatacttgcacctaacaattattatccttctaagaacttggcgccaatcgcattaaatgcacagaaggatttcagttctactccaattacaatgaattacaatcaatttcttgagttggaaaagacgaaacagttaagattagatacggaccaggtatatgggaacatagcaacatacaattttgaaaatggaagagtgagggtggacacaggctcgaactggagtgaagtgttaccgcaaattcaagaaacaactgcacgtatcatttttaacggaaaggatttaaacctggtagaaaggcggatagcggccgttaatcctagtgatccattagaaacgactaagccggacatgacattaaaggaggcccttaagatagcatttggatttaacgaaccgaatggaaacttacagtatcaggggaaggacataaccgaatttgattttaatttcgatcaacaaacatctcaaaacatcaagaaccagttagcggagttaaacgcaactaacatatatactgtattagataagatcaagttaaatgcaagaatgaacattttaataagagacaagcgtttccattatgatagaaataacatagcagttggggcggacgagtcagtagttaaggaggctcatagagaagtaattaactcgtcaacagagggattattgttaaacattgataaggacataaggaagatattatcaggttatattgtagaaattgaagatactgaagggcttaaggaagttataaacgacagatatgatatgttgaatatttctagtttacggcaagatggaaagacatttatagacttcaagaagtataatgataagttaccgttatatataagtaatcccaattataaggtaaatgtatatgctgttactaaggaaaacactattattaatcctagtgagaatggggatactagtaccaacgggatcaagaagattttaatcttctctaagaagggctatgagataggataa

SEQ ID NO: 28 - protein, 1022 aa, Artificial
  Protein expressed by modified rabies virus N-Fu-PA63
MDADKIVFKVNNQVVSLKPEIIVDQYEYKYPAIKDLKKPCITLGKAPDLNKAYKSVLSGMSAAKLNPDDVCSYLAAAMQFFEGTCPEDWTSYGIVIARKGDKITPGSLVEIKRTDVEGNWALTGGMELTRDPTVPEHASLVGLLLSLYRLSKISGQNTGNYKTNIADRIEQIFETAPFVKIVEHHTLMTTHKMCANWSTIPNFRFLAGTYDMFFSRIEHLYSAIRVGTVVTAYEDCSGLVSFTGFIKQINLTAREAILYFFHKNFEEEIRRMFEPGQETAVPHSYFIHFRSLGLSGKSPYSSNAVGHVFNLIHFVGCYMGQVRSLNATVIAACAPHEMSVLGGYLGEEFFGKGTFERRFFRDEKELQEYEAAELTKTDVALADDGTVNSDDEDYFSGETRSPEAVYTRIMMNGGRLKRSHIRRYVSVSSNHQARPNSFAEFLNKTYSSDSRTTMSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKADENQLSQILAPNNYYPSKNLAPIALNAQKDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNARMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 29 - DNA, 2073 bp, Artificial
  Nucleic acid of modiefied rabies virus expressing Nfu-GFP
atggatgccgacaagattgtattcaaagtcaataatcaggtggtctctttgaagcctgagattatcgtggatcaatatgagtacaagtaccctgccatcaaagatttgaaaaagccctgtataaccctaggaaaggctcccgatttaaataaagcatacaagtcagttttgtcaggcatgagcgccgccaaacttaatcctgacgatgtatgttcctatttggcagcggcaatgcagttttttgaggggacatgtccggaagactggaccagctatggaattgtgattgcacgaaaaggagataagatcaccccaggttctctggtggagataaaacgtactgatgtagaagggaattgggctctgacaggaggcatggaactgacaagagaccccactgtccctgagcatgcgtccttagtcggtcttctcttgagtctgtataggttgagcaaaatatccgggcaaaacactggtaactataagacaaacattgcagacaggatagagcagatttttgagacagccccttttgttaaaatcgtggaacaccatactctaatgacaactcacaaaatgtgtgctaattggagtactataccaaacttcagatttttggccggaacctatgacatgtttttctcccggattgagcatctatattcagcaatcagagtgggcacagttgtcactgcttatgaagactgttcaggactggtatcatttactgggttcataaaacaaatcaatctcaccgctagagaggcaatactatatttcttccacaagaactttgaggaagagataagaagaatgtttgagccagggcaggagacagctgttcctcactcttatttcatccacttccgttcactaggcttgagtgggaaatctccttattcatcaaatgctgttggtcacgtgttcaatctcattcactttgtaggatgctatatgggtcaagtcagatccctaaatgcaacggttattgctgcatgtgctcctcatgaaatgtctgttctagggggctatctgggagaggaattcttcgggaaagggacatttgaaagaagattcttcagagatgagaaagaacttcaagaatacgaggcggctgaactgacaaagactgacgtagcactggcagatgatggaactgtcaactctgacgacgaggactacttttcaggtgaaaccagaagtccggaggctgtttatactcgaatcatgatgaatggaggtcgactaaagagatctcacatacggagatatgtctcagtcagttccaatcatcaagcccgtccaaactcattcgccgagtttctaaacaagacatattcgagtgactcacgtacggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 30 - protein, 690 aa, Artificial
  Protein expressed by modified rabies virus Nfu-GFP
MDADKIVFKVNNQVVSLKPEIIVDQYEYKYPAIKDLKKPCITLGKAPDLNKAYKSVLSGMSAAKLNPDDVCSYLAAAMQFFEGTCPEDWTSYGIVIARKGDKITPGSLVEIKRTDVEGNWALTGGMELTRDPTVPEHASLVGLLLSLYRLSKISGQNTGNYKTNIADRIEQIFETAPFVKIVEHHTLMTTHKMCANWSTIPNFRFLAGTYDMFFSRIEHLYSAIRVGTVVTAYEDCSGLVSFTGFIKQINLTAREAILYFFHKNFEEEIRRMFEPGQETAVPHSYFIHFRSLGLSGKSPYSSNAVGHVFNLIHFVGCYMGQVRSLNATVIAACAPHEMSVLGGYLGEEFFGKGTFERRFFRDEKELQEYEAAELTKTDVALADDGTVNSDDEDYFSGETRSPEAVYTRIMMNGGRLKRSHIRRYVSVSSNHQARPNSFAEFLNKTYSSDSRTVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 31 - DNA, 1575 bp, Artificial
  Nucleic acid of rabbies virus G
atggttcctcaggctctcctgtttgtaccccttctggtttttccattgtgttttgggaaattccctatttacacgataccagacaagcttggtccctggagtccgattgacatacatcacctcagctgcccaaacaatttggtagtggaggacgaaggatgcaccaacctgtcagggttctcctacatggaacttaaagttggatacatcttagccataaaagtgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtggcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtctcgatgcaaacatcaaatgaaaccaaatggtgccctcccgataagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcagaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtaa

SEQ ID NO: 32 - protein, 524 aa, Artificial
  Protein expressed by modified rabies virus G protein
MVPQALLFVPLLVFPLCFGKFPIYTIPDKLGPWSPIDIHHLSCPNNLVVEDEGCTNLSGFSYMELKVGYILAIKVNGFTCTGVVTEAETYTNFVGYVTTTFKRKHFRPTPDACRAAYNWKMAGDPRYEESLHNPYPDYRWLRTVKTTKESLVIISPSVADLDPYDRSLHSRVFPSGKCSGVAVSSTYCSTNHDYTIWMPENPRLGMSCDIFTNSRGKRASKGSETCGFVDERGLYKSLKGACKLKLCGVLGLRLMDGTWVSMQTSNETKWCPPDKLVNLHDFRSDEIEHLVVEELVRKREECLDALESIMTTKSVSFRRLSHLRKLVPGFGKAYTIFNKTLMEADAHYKSVRTWNEILPSKGCLRVGGRCHPHVNGVFFNGIILGPDGNVLIPEMQSSLLQQHMELLESSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

SEQ ID NO: 33 - DNA, 867 bp, Artificial
  Nucleic acid of modified rabies virus expressing D4-E51
atggttcctcaggctctcctgtttgtaccccttctggtttttccattgtgttttgggaaattccctactagggacaagcgtttccattatgatagaaataacatagcagttggggcggacgagtcagtagttaaggaggctcatagagaagtaattaactcgtcaacagagggattattgttaaacattgataaggacataaggaagatattatcaggttatattgtagaaattgaagatactgaagggcttaaggaagttataaacgacagatatgatatgttgaatatttctagtttacggcaagatggaaagacatttatagacttcaagaagtataatgataagttaccgttatatataagtaatcccaattataaggtaaatgtatatgctgttactaaggaaaacactattattaatcctagtgagaatggggatactagtaccaacgggatcaagaagattttaatcttctctaagaagggctatgagataggacctagagaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaaggttaactatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtaa

SEQ ID NO: 34 - protein, 288 aa, Artificial
  Protein expressed by modified rabies virus D4-E51
MVPQALLFVPLLVFPLCFGKFPTRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIGPRESSVIPLVHPLADPSTVFKDGDEAEDFVEVHLPDVHNQVSGVDLGLPNWGKVNYVLLSAGALTALMLIIFLMTCCRRVNRSEPTQHNLRGTGREVSVTPQSGKIISSWESHKSGGETRL